(19) United States
(12) Patent Application Publication
(10) Pub. No.: US 2023/0165501 A1
(43) Pub. Date: Jun. 1, 2023

(54) SYSTEM AND METHODS FOR CONTACTLESS MONITORING OF HEART MUSCLE ACTIVITY AND IDENTIFYING MEDICAL CONDITIONS BASED ON BIOPOTENTIAL SIGNALS

(71) Applicant: EASYG LLC, Beverly Hills, MI (US)

(72) Inventors: Alireza Moghaddambagheri, Vancouver BC (CA); Thomas Evans, Beverly Hills, MI (US); Derrick Redding, Ann Arbor, MI (US)

(21) Appl. No.: 17/916,670

(22) PCT Filed: Apr. 5, 2021
(86) PCT No.: PCT/US2021/025611

§ 371 Date: Oct. 3, 2022

Related U.S. Application Data
(60) Provisional application No. 63/004,868, filed on Apr. 3, 2020.

(51) Int. Cl. A61B 5/26; A61B 5/282; A61B 5/00; A61B 5/346

(52) U.S. Cl. CPC A61B 5/26; A61B 5/282; A61B 5/6891; A61B 5/6892; A61B 5/7246; A61B 5/346; A61B 2503/045; A61B 2562/0209; A61B 2560/0468

(57) ABSTRACT

The present teachings relate to monitoring the condition of a subject with a contactless system for sensing biopotential signals comprising: a support surface; one or more inner layers; a plurality of contactless electrode units within the one or more inner layers; one or more outer layers; and wherein the plurality of contactless electrode units are arranged in an inner shape within an outer shape such that the contactless electrode units form the vertices of the inner shape and the outer shape. The method includes the steps of: providing a support surface having one or more sensing devices embedded therein; positioning the subject at least partially on the support surface; acquiring data from an electrocardiograph reading on the subject for a predetermined amount of time; outputting the data of the step (c); and analyzing the data of the step (c), by identifying one or more biomarkers consistent with a disease condition.

### CROSS REFERENCE TO RELATED APPLICATIONS

Applicant claims the benefit of U.S. Provisional Application No. 63/004,868 filed on Apr. 3, 2020, the contents of which are incorporated herein by reference in their entirety.

### FIELD

The present teachings relate to a system and method for monitoring heart muscle activity of an individual in a variety of settings while avoiding direct contact with the skin of the individual. The present teachings may relate to a system having electrodes which are compatible with a large portion of the population, including infants, children, adolescents, adults, or a combination thereof, while avoiding customized placement on the individual. The present teachings relate to a system and method for monitoring heart muscle activity of a patient to detect and/or monitor the presence of one or more medical conditions, including viruses.

### BACKGROUND

Conventional electrocardiograph systems typically require placement of three to ten electrodes placed directly on the skin of a subject to detect electrical activity and associated biopotential signals. These electrodes are in electrical communication with a cardiac monitor which receives the biopotential signals from the electrodes. These electrodes are typically in the form of disposable electrodes with peel-and-stick adhesive that are adhered to specific vantage points on an individual's body, such as origins or ends of limbs. These conventional systems require disposal of the electrodes after each use, may cause irritation or other allergic reactions of individuals, require storage of supplies, and require the time for placement on each individual.

A main challenge with conventional electrocardiograph is the extensive time to appropriately place each electrode on an individual. Individual placements allow for customized placement of the electrodes based on an individual's anthropometry. Individual placement also requires disposable of the electrodes after each use. Each electrode is associated with its own wire and multiple electrodes adhered to an individual also requires maneuvering, avoiding tangling, and detangling of these wires. Further, due to the complexity of these conventional electrocardiograph systems, their use is typically limited to medical settings. What is needed is an electrocardiograph system which can accommodate a large size of the population while the electrodes remain statically affixed to a support surface. What is needed is an electrocardiograph system which can be reused to avoid waste. What is needed is an electrocardiograph system which can be used in a multitude of settings, from hospital settings to residential homes to vehicles.

Another challenge with conventional electrocardiograph systems is the ability to use with infants. Often, infants under care in neonatal intensive care units are continuously monitored with electrocardiograph systems. These systems are customized for infants by using neonatal and pediatric electrodes which use smaller electrodes, different adhesives that minimize irritation on delicate skin of a newborn, and apply reduced pressure to the skin. These neonatal and pediatric electrodes can be even more problematic for premature infants, such as below 32 weeks gestational age. These conventional electrodes may limit access to an infant's torso for day-to-day care, still cause irritation on the infant's skin, further exacerbate issues of limited space within the incubator, the adhesive may easily peel and unstick due to the humidity within a humidified incubator, and the wires may easily tangle with other medical devices within an incubator. What is needed is an electrocardiograph system useful with infants, such as those in neonatal intensive care units, located within incubators, or both. What is needed is an electrocardiograph system which avoids at least some direct contact and use of adhesive on an infant's delicate skin. What is needed is an electrocardiograph system compatible with the environment within an incubator, including increased humidity and limited space. What is needed is an electrocardiograph that can accommodate infants of varying sizes, an infant during periods of growth, or both.

Viruses in humans are generally detected via culture methods and nonculture methods. Culture methods include viral cultures. A viral culture typically tests samples, such as from swabs or blood, by placing the sample with a cell type that a virus can infect. If the cells show changes, the culture is positive for the particular virus being tested for. Nonculture methods include rapid testing methods which still require a sample, such as from a swab or blood, and place the samples in rapid diagnostic testing machines (e.g., polymerase chain reaction devices), which may not be immediately present or available at the site of testing. The rapid diagnostic testing machines directly detect the presence or absence of an antigen of a particular virus. While these tests can accurately detect the presence of a virus, they can be time-consuming if awaiting a cell culture result and even when trying to test multiple individuals in a limited time frame. Additionally, these tests can be invasive, causing patient discomfort, based on the sample collection method used, such as a nasal swab, throat swab, or blood sample. For COVID-19, polymerase chain reaction can take one day or even more to receive results if a test is positive or negative for the virus. Onsite rapid detection tests are currently under development for detection of COVID-19 and predicted to take between 5-15 minutes per individual. Additionally, these tests often are not being given until an individual is symptomatic due their processing time and not burdening the testing system (e.g., in the instance of an epidemic or pandemic). Thus, what is needed is a method for faster diagnosis that may even be able to detect the presence of the virus before the onset of symptoms.

Animal trials have found that EKG signals may detect the presence of a virus before symptoms or other physiological symptoms are onset. For example, animal trials with primates for Ebola detection and with rabbits for Coronavirus detection found that certain biomarkers in EKG signals showed a change 40 hours before developing fever or other symptoms. As taught in the Journal of Electrocardiology, 15 EKG biomarkers were analyzed in rabbits infected with Coronavirus. Of the 15 biomarkers, three biomarkers showed changes in the sub-acute phase: T-wave depression (95% of rabbits), Sinus Tachycardia (90% of rabbits), and QTc prolongation (89% of rabbits). “ECG Changes After Rabbit Coronavirus Infection.” Journal of Electrocardiology. Vo. 32. No. 1. 1999. “Detecting Pathogen Exposure During the Non-Symptomatic Incubation Period Using Physiological Data” published by the Massachusetts Institute of Technology Lincoln Laboratory. Notwithstanding, there is still a need to understand what biomarkers may be associated with certain medical conditions, including infections and diseases, in humans, and even animals, along with a system and method for detecting the presence of those biomarkers.

Generally, electrocardiograph tests can be both an intimate and time-consuming process. Patients may be asked to partially undress to allow for direct contact and adhesion of electrodes onto their skin. Electrocardiograph tests also require physical interaction and contact between the healthcare workers attending to the patient and the patient undergoing an exam. This proximity may expose either party to an illness, and the risk of transmission from one party to the other. In particular, health care workers may face more risks to infection while applying and removing EKG electrodes and leads from a patient. For example, administering a traditional contact electrocardiograph poses a risk with the health care workers being exposed to the COVID-19 virus.

Newer viral infections, such as COVID-19, have been reported as presenting higher fatality rates than other viral infections. In particular, according to some reported information, patients with cardiovascular disease have the highest average fatality rate at 10.5% from COVID-19 as compared to other comorbid patients. Additionally, 16.7% of patients hospitalized for COVID-19 develop arrythmia. Often, patients are not being medically attended to until after the onset of symptoms and severity worsens, such as after the onset of pneumonia, when certain therapies may no longer be suitable for treatment.

To help cure an individual from COVID-19, chloroquine and hydroxychloroquine have started to be used. These drugs, and others, are known to cause drug-induced arrhythmias from prolonged QTc intervals. In rare cases, the drug-induced arrhythmia can cause a patient's heart to completely stop resulting in death. An electrocardiograph detecting one or more biopotentials, such as QTc, may be useful in screening patients as good candidates for experimental medicine trials, before commencing a medicine routine to treat a medical condition, and/or the like. What is needed is a system and method for detecting one or more biopotentials without the need to be in close contact with the patient. What is needed is a method for detecting one or more biopotentials for screening patients. What is needed is a method for monitoring patients for changes in biopotentials deviating from the individual's baseline and/or indicating of a medical condition occurring while participating in a medical trial, undergoing medical treatment, or the like.

### SUMMARY

The present disclosure relates to methods, devices and systems. The devices herein may find particular use in accommodating a wide array of the population for sensing biopotential signals without requiring direct contact with the individual's skin and being reusable across a variety of settings and number of subjects (e.g., humans). The methods, devices and systems, though having other application (as the teachings herein will reveal) may share a common objective of acquisition of data from an electrocardiograph reading on the subject (e.g., human) for a predetermined amount of time and analysis of the data for identifying the presence of one or more biomarkers (i.e., coronary biomarkers, such as those pertaining to heart muscle activity) consistent with a medical condition, such as viral infection or other disease. The methods, devices and systems, are particularly useful for early detection of the medical condition. Thus, in general, the methods, devices and systems described herein may be employed prior to (e.g., at least 72, 48, 36, 12, 8, 4, or 2 hours before) the onset of objectively detectable symptoms (e.g., before a patient temperature reading departs (such as by at least 0.5, 1, 1.5 or 2° C.) from a typical normal temperature of 37° C.), and/or other identified symptoms (e.g., sore throat, persistent cough, difficulty breathing, pain, etc.).

The present disclosure relates to a contactless system for sensing biopotential signals from a subject comprising: a) a support surface; b) one or more inner layers including one or more deflecting materials; c) a plurality of contactless electrode units within the one or more inner layers, the one or more electrode units including one or more capacitive sensors; d) one or more outer layers located about the one or more inner layers; and wherein the plurality of contactless electrode units are arranged in an inner shape within an outer shape such that the contactless electrode units form the vertices of the inner shape and the outer shape.

The present disclosure relates to a method of monitoring the condition of a subject (e.g., human), comprising the steps of: a) providing a support surface (e.g., a chair, pad, or bed) having one or more sensing devices of an electrocardiograph device embedded therein and/or providing the one or more sensing devices of the electrocardiograph device; b) positioning the subject (e.g., human) (e.g., a medical worker or a patient) at least partially on the support surface (e.g., in a sitting position opposing the one or more sensing devices in a seatback) and/or positioning the one or more sensing devices on the subject; c) acquiring data from an electrocardiograph reading on the subject (e.g., human) for a predetermined amount of time (e.g., from about 5 sec to about 2 minutes); d) outputting the data of the step (c) (e.g., into an electronic memory storage device); and e) analyzing the data of the step (c), by identifying one or more biomarkers consistent with a medical condition.

The present disclosure relates to a method of machine learning to identify one or more biomarkers indicative of a medical condition comprising: a) acquiring data from an electrocardiograph reading on a subject (e.g., human) for a predetermined amount of time (e.g., from about 5 sec to about 2 minutes); b) outputting the data of the step (a) (e.g., into an electronic memory storage device); c) applying one or more data labels identifying a health status of the subject (e.g., human); and d) analyzing the data to find a correlation between the data and the one or more data labels to determine the one or more biomarkers indicative of the medical condition.

The present disclosure also relates to a device that is used for acquiring data from a patient, to computer programs for performing teachings as will be described herein.

Though the present application is described primarily for use to investigate a condition of a human, the present disclosure also has veterinary applicability for animals.

The present teachings may provide a device which allows for contactless administration of an electrocardiograph. The present teachings may provide a support surface which allows for electrode units to be statically affixed therein. The present teachings may provide a support surface compatible with at least 80% of the population, 90% of the population, or even 100% of the population. The support surface may be compatible with heights varying from a 5′^{h} percentile female to a 95^{th} percentile male. The support surface may be reusable across a number of patients. The present teachings provide a support surface which may be compatible with infants, infant incubators, pediatric patients, pediatric beds, or any combination thereof. The present teachings may provide a support surface adapted to accommodate prematurely born infants, infants under neonatal intensive care, children under pediatric care, or a combination thereof. The present teachings may present an unconventional approach to detecting biopotential signals by using a standard support surface with static non-contract electrode units therein able to accommodate a wide array of the population as opposed to custom electrode placement per patient. The present teachings may provide a method of collecting biopotential data in fewer steps as compared to a traditional electrocardiograph. The present teachings may provide a method of quickly determining if a subject has a medical condition, such as being positive for a disease condition. The method may be able to be quickly administered and repeated for a number of subjects. The present teachings may provide a method for quickly determining one or more biomarkers indicative of one or more medical conditions. The present teachings may provide a system and/or method which use substantially less consumables than traditional culture methods, nonculture methods, electrocardiograph tests, and the like. The present teachings may provide an unconventional approach at detecting the presence of medical conditions by using biopotential signals and associated data to determine a medical condition, as opposed to more invasive testing, or only using the biopotential signal data for detecting the presence of traditional cardiac medical conditions.

### BRIEF DESCRIPTION OF DRAWINGS

FIG. 1 illustrates a user positioned on a support surface.

FIG. 2 illustrates a support surface.

FIG. 3 illustrates a support surface.

FIG. 4 illustrates a support surface.

FIG. 5 illustrates a plurality of sensing devices and an inner layer of a support surface.

FIG. 6 illustrates a schematic of a plurality of sensing devices of a support surface.

FIG. 7A illustrates a cross-section of a support surface through a plurality of sensing devices.

FIG. 7B illustrates a cross-section of a support surface through a plurality of sensing devices.

FIG. 8 illustrates a schematic of a system.

FIG. 9 illustrates a system integrated with a neonatal incubator.

FIG. 10 illustrates a plurality of ports of a computing platform as a monitoring device.

FIG. 11 illustrates a support surface with position indicators.

FIG. 12A illustrates a cross-section of a support surface through section A-A of FIG. 11.

FIG. 12B illustrates a cross-section of a support surface through section A-A of FIG. 11.

FIG. 13 illustrates placement of electrode units within a support surface.

FIG. 14 illustrates a schematic of electrode units within a support surface.

FIG. 15 illustrates a flow of a subject using a user interface of a system.

FIG. 16 illustrates a method of machine learning to identify one or more biomarkers indicative of a medical condition.

FIG. 17 illustrates a method of detecting and identifying the presence and/or absence of a medical condition.

FIG. 18 illustrates a network of a machine learning system.

FIG. 19 illustrates a network of a machine learning system.

FIG. 20 illustrates a method for preparing data for machine learning to identify one or more biomarkers indicative of a medical condition.

FIG. 21 illustrates a method of machine learning to identify one or more biomarkers indicative of an infection and/or disease.

### DETAILED DESCRIPTION

The present teachings meet one or more of the above needs by the improved devices and methods described herein. The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

System

The present teachings may relate to a system and apparatus for monitoring heart muscle activity of an individual, including at least a first electrode unit (e.g., sensing device) for receiving a first signal indicative of electrical activity at a first location on a body of the individual, and a second electrode unit (e.g., sensing device) for receiving a second signal indicative of electrical activity at a second location on the body of the individual. The first and second electrodes may be contact or non-contact EKG sensors. The system may include further electrodes, such a third electrode for receiving a third signal indicative of electrical activity at a second location on a body of the individual. The system may be multiplied in size or capacity, such that there are five electrode units, six electrode units, nine electrode units, twelve electrode units, or even more. The electrical signals gathered from the electrodes can be used in early detection of medical conditions, such as viruses and other diseases. A machine learning system to feed EKG data of individuals with or without known medical conditions (e.g., infected and non-infected patients with certain viruses or other diseases) to develop an algorithm that can predict the presence of a medical condition before signs or symptoms occur.

The present teachings relate to a system including an electrocardiograph device. The electrocardiograph device may function to detect one or more biopotentials (e.g., physiological electrical activity) in an individual, save the detected biopotentials as data, analyze the data, or any combination thereof. The biopotentials may be heart muscle activity of a subject. A subject may be a human or other animal. A human may include an infant, child, adolescent, adult, or a combination thereof. The device may include one or more support surfaces, sensing devices, conductors, electronic processors, electronic memory storage devices, user interfaces, the like, or any combination thereof. The electrocardiograph device may include one or more components as described in U.S. Pat. No. 10,182,732; US Patent Application Publication No.: 2019/0090747; PCT Application Nos. PCT/US2019/063403 and PCT/US2019/063410; and U.S. Application No. 62/772,248 filed 28 Nov. 2018, all of which are incorporated herein by reference in their entirety.

The system may include a support surface. The support surface may function to allow a subject (e.g., human or other animal) to rest thereon and be in sensing communication with one or more sensing devices of an electrocardiograph device, may house one or more sensing devices, may maintain one or more sensing devices distanced from and not in contact with a subject, or any combination thereof. The support surface may be suitable for one or more settings. One or more settings may include one or more hospitals, vehicles, residences, commercial buildings, the like, or any combination thereof. One or more hospital settings may include the intensive care unit (ICU), intensive therapy unit (IT), neonatal intensive care unit (NICU), critical care unit (CC), hospice, the like, or a combination thereof within a hospital. One or more residences may include individual homes, assisted living homes, the like, or a combination thereof. The support surface may be any surface suitable for having an individual rest thereon, have one or more sensing devices disposed therein, having one or more conductive materials disposed therein, allow for an individual to be in sensing distance from one or more sensing devices, or any combination thereof. The support surface may include one or more outer layers, one or more sensing devices, one or more inner layers, one or more conductive components, the like, or a combination thereof. The support surface may allow for an individual to lay their back against the support surface in a seated, lying, standing, and/or other position.

The support surface may include and/or be part of a chair, bed, stretcher, gurney, incubator, stroller, wheelchair, a pad for placement on a secondary support surface, the like, or a combination thereof. The secondary support surface may be a chair, bed, stretcher, gurney, stroller, wheelchair, incubator, floor, table, or any other surface. The secondary support surface may be another user. For example, another user may hold the support surface against a subject. As a more detailed example, an adult may hold a hand-sized support surface against the backside of an infant, such as while being held. The secondary support surface may be vertical, horizontal, a position therebetween, or a combination thereof. The support surface may be integrated into and/or disposed onto, at least a portion of the secondary support surface. For example, the support surface may be located against a backrest of a chair. For example, the support surface may be integrated into a backrest of a chair. As another example, the support surface may rest on a hospital bed. And as a further example, the support surface may rest on a base of an incubator.

The support surface may have a shape suitable for cooperating with and/or being integrated into a secondary support surface, having a subject rest thereon, housing one or more sensing devices, housing one or more conductive materials, or any combination thereof. The support surface may have one or more profile shapes. A profile shape may be a two-dimensional shape at a face or cross-section of the support surface. One or more profile shapes may include a front profile, side profile, or both. A front profile may be the two-dimensional shape of a front face. A side profile may be the two-dimensional shape of one or more side faces. One or more profile shapes may be substantially and/or partially square, rectangular, triangular, circular, ovular, elliptical, the like, or any combination thereof. One or more profile shapes may be the same or differ from one or more other profile shapes. For example, one or more front profile shapes may be substantially rectangular. As another example, one or more front profile shapes may have a pill shape, such as substantially rectangular with circular ends. The support surface may have a shape such that it includes a front face opposing a rear face. A front face may be the face which is adjacent to and facing toward a subject. A rear face may be the face which is adjacent to a secondary support surface, opposite the front face, or both. The support surface may include a top face opposing a bottom face. The front and/or rear faces may extend from the top face to the bottom face. The bottom face may function to rest on a resting surface of a secondary support surface. The support surface may include opposing side faces. As an example, one or more side profile shapes may be substantially rectangular. The support surface may have an overall shape which is substantially planar (e.g., flat), nonplanar, or both. Nonplanar may allow for the support surface to be at least partially reciprocal with the backside of a subject, a secondary support surface, or both. A reciprocal contour in the support surface may allow for the front face of the support surface to better contour to a backside of a subject, have one or more sensing device in a better position relative to the subject, or both. Nonplanar may indicate where to locate a subject on a front face. Nonplanar may allow for the support surface to be biased. The bias may be toward and/or away from the backside of the subject. The bias may be created by one or more inner layers. The bias may allow for a subject to deflect the front face when resting thereon, maximize contact with their backside and a front face, or both. Nonplanar may include convex, concave, or both.

The support surface may have a size suitable for cooperating with and/or being integrated into a secondary support surface, able to have a subject rest thereon, house one or more sensing devices, house one or more conductive materials, or any combination thereof. The support surface may have a shape and/or size suitable for having a length of a backside of a torso thereon, a length of an entire body of a subject thereon, or both. The support surface may have a length equal to or greater than a length of a torso, a height of an individual, or both. The length of a torso may be defined as the distance from the top edge of the shoulders to the top crest of the hip bone of a human. The length and/or height of an individual (i.e., subject) may be a length from the top of the head to the bottom of the heels of the individual. The size of the support surface may accommodate varying sized subjects. Varying sized may include different heights, weights, widths, ages, and the like. The size of the support surface may accommodate infants, children, adolescents, adults, or a combination thereof. The size of the support surface may accommodate infants aged newborn (including premature infants) to 1 year old. The size of the support surface may accommodate prematurely born infants. The size of the support surface may accommodate 80% or greater, 85% or greater, 90% or greater, or even 95% or greater of the adult population. The size of the support surface may accommodate a 5^{th} percentile female to a 95% percentile male. A single support surface may be able to accommodate a premature infant (e.g., 30 weeks gestation) to an adult male (e.g., 95^{th} percentile male). Different support surfaces may be sized to accommodate different age brackets. The support surface may have a height measured as a distance from a bottom face to a top face. The support surface may have a height of about 10 cm or greater, about 15 cm or greater, about 20 cm or greater, about 25 cm or greater, about 30 cm or greater, about 40 cm or greater, about 50 cm or greater, about 55 cm or greater, about 60 cm or greater, about 65 cm or greater, about 70 cm or greater. The support surface may have a height of about 200 cm or less, about 190 cm or less, about 185 cm or less, about 180 cm or less, about 170 cm or less, about 160 cm or less, or even about 150 cm or less. For example, a support surface intended for use with an infant in an incubator may have a height of about 60 cm to about 90 cm. For example, a support surface intended for use as a backrest with adults may have a height of about 60 cm to about 100 cm. The support surface may have a width of about 10 cm or greater, about 15 cm or greater, about 20 cm or greater, about 25 cm or greater, about 30 cm or greater, about 40 cm or greater, about 50 cm or greater, or even about 60 cm or greater. The support surface may have a width of about 100 cm or less, about 90 cm or less, about 80 cm or less, or even 70 cm or less. For example, a support surface intended for use with an infant in an incubator may have a width of about 35 cm to about 50 cm. For example, a support surface intended for use as a backrest with adults may have a width of about 45 cm to about 60 cm.

The support surface may be transportable and/or statically affixed. Transportable may mean with or free of any propulsion means (e.g., free of any motor). Transportable may mean being able to be moved independently from a secondary support surface, setting, or both. Statically affixed may mean the support surface is integrated into a secondary support surface, affixed to a vehicle, the like, or a combination thereof. Statically affixed may mean that the support surface is unable to be separated from a secondary support surface, remains within the same setting, or both. Transportable may allow for the support surface to be easily used with varying secondary support surfaces, in varying settings, in varying locations, or any combination thereof. For example, a transportable secondary surface may be located on a variety of chairs to accommodate different subjects. As another example, a support surface may be statically affixed within a seat of a vehicle by being integrated into the seat (e.g., chair).

The support surface, secondary support surface, or both may be fixed or adjustable. Adjustable may mean that one or more angles of the support surface may be adjustable. Fixed may mean that the overall shape of the support surface stays substantially the same. Adjustment may be achieved mechanically, by a motor, or both. A support surface may be integrated into a secondary support surface (e.g., chair, bed) such that the support surface includes a back rest, seat rest, leg rest, or combination thereof. Adjustment may allow for one or more angles between a back rest, seat rest, leg rest, or a combination thereof to be adjusted relative to one another. For example, the support surface may be integrated into a bed (e.g., mattress) or lay on top of a bed which is able to have angles adjusted for the upper and lower body. As another example, the support surface may be integrated into a seat of a vehicle which includes multi-way adjustment.

The support surface may be freestanding, supported by another structure (e.g., secondary support surface), or both. The support surface, secondary support surface, or both may be free of or include one or more legs, wheels, or both. The support surface, secondary support surface, or both may be attached or free of attachment to one or more fixed surfaces (e.g., a vehicle interior, chair, mattress, incubator).

The support surface may include one or more outer layers. The one or more outer layers may include the one or more layers a subject may come into contact with, rest on, or both. The one or more outer layers may at least partially house and/or encapsulate one or more inner layers, other outer layers, or both. One or more outer layers may provide an exterior surface of the support surface. The one or more outer layers may protect one or more inner layers, provide a barrier between one or more sensing devices and a subject, or both. The one or more outer layers may include one or more permanent layers, temporary layers, or both. One or more permanent layers may not be removable relative to one or more inner layers. One or more temporary layers may be removable relative to one or more inner layers, permanent layers, or both. The one or more layers may be disposable, biodegradable, one-time use, reusable, cleanable, the like, or any combination thereof. One or more permanent layers may be waterproof, easily disinfected, reusable, or a combination thereof. One or more temporary layers may be disposable, one-time use, biodegradable, reusable, and/or washable barrier. One or more temporary layers may cooperate with one or more permanent layers, inner layers, or both. One or more temporary layers may be disposed between a permanent layer and an individual. One or more temporary layers may be in direct contact and at least partially surround one or more inner layers.

One or more outer layers may include one or more outer materials. One or more outer materials may be suitable for use in one or more settings. The one or more outer materials may include one or more organic materials, inorganic materials, or both. The one or more outer materials may include leather, suede, polyurethane, polypropylene, thermoplastic polyurethane, vinyl, polyvinyl chloride, cotton, polyester, linen, paper, the like, or a combination thereof. The one or more outer layers may include one or more woven and/or nonwoven fabrics. For example, a suitable permanent layer may be leather, suede, polyvinyl chloride, and the like. For example, a suitable temporary layer may include disposable non-woven paper, a washable fabric cover, or both. For example, a temporary layer may include a portion of a roll of poly-paper that is temporarily located on a permanent outer layer. One or more outer materials may aid in one or more sensing devices receiving one or more signals from the individuals. The one or more outer materials may provide for electrical conductivity at a level less than, equal to, or greater than that of human skin. Electrical conductivity may refer to surface electrical resistance. Having electrical conductivity about equal to or greater than that of human skin may aid in transmitting one or more biopotential signals from a subject to the one or more sensing devices. One or more outer materials may include one or more added materials. The one or more added materials may provide for reinforcement, electrical conductivity, elasticity, or any combination thereof.

The one or more inner layers may function as cushion for supporting and providing comfort to an individual, contouring to the shape of a body of an individual resting thereon, housing one or more sensing devices, providing a bias, or any combination thereof. The one or more inner layers may include one or more deflecting materials, one or more conductive sheets, or both. The one or more deflecting materials may be sensitive to pressure and/or temperature from a body of a subject, mold to the shape of the portion of the body resting thereon, allow the support surface to contour to the subject's body, or any combination thereof. The one or more conductive sheets may provide for one or more conductive paths. The one or more inner layers may include one or more inner materials. The one or more inner materials may function to dampen movement of a body which may interfere with the biopotential signals. Movement of the body may include ballistic movement caused by the flow of blood through the subject's body. This ballistic movement can result in ballistic effects, such as pulse artefact which is picked up by the electrode unit and amplifier. The one or more inner materials may be flexible, elastomeric, water resistant, the like, or a combination thereof. The one or more inner materials may include one or more foams, sponges, rubbers, springs, the like, or a combination thereof. One or more foams may be open cell, closed cell, or both. One or more inner materials may include one or more polymers. One or more inner materials may include polyurethane, polyethylene terephthalate, polyester, polyvinyl chloride, nitrile, silicone, plastazote, vegetal cellulose, neoprene, sorbothane, ethylene propylene diene monomer, the like, or any combination thereof. The one or more inner layers may include one or more wells therein. The one or more wells may house sensing devices. The one or more wells may allow for one or more proximate surfaces (e.g., outward/front facing surface) of one or more sensing devices to be substantially flush with, below, or even above one or more front facing surfaces of one or more inner layers. The one or more wells may be formed prior to placement of one or more sensing devices therein, while locating the one or more sensing devices therein (e.g., via over molding), or both. The one or more inner layers may cooperate with the one or more outer layers to retain one or more sensing devices therebetween. One or more sensing layers may be in direct contact or indirect contact with one or more outer layers, inner layers, or both.

The system may include one or more sensing devices. The one or more sensing devices may function to detect one or more biopotential signals, vital signals, physiological signals, the like, or any combination thereof. The one or more sensing devices may be wired, wireless, or both. Wired may mean that the one or more sensing devices are in direct electrical communication with an electronic processor, memory storage device, user interface, or a combination thereof via one or more wires such that signals received by the one or more sensing devices are transmitted via the one or more wires. Wireless may mean that the one or more sensing devices are not physically connected to the electronic processor, memory storage device, user interface, or a combination thereof and may transmit the signals received by one or more wireless modes of communication. The one or more sensing devices may be one or more contact sensing devices, non-contact sensing devices, or both. Contact may mean that the one or more sensing devices are placed in direct contact with an individual. Contact may mean traditional electrodes that are adhered to the skin of an individual. Non-contact may mean one or more sensing devices which avoid direct contact with the skin of an individual. One or more non-contact sensing devices may be suitable such that an individual may still be able to wear clothing. Clothing may include a medical gown, personal protective equipment, a shirt or blouse, a jacket (e.g., medical jacket), the like, or any combination thereof. The individual may be able to wear one or more layers of clothing while a non-contact sensing device is still able to detect one or more signals from the individual. The one or more sensing devices may be placed on, embedded in, or both, one or more support surfaces. The one or more sensing devices may be located within one or more wells of one or more inner layers. The one or more sensing devices may be located adjacent to one or more outer layers, embedded within one or more inner layers, or both. One or more sensing devices may include any device capable of detecting and measuring one or more biopotential signals, vital signals, physiological signals, or any combination thereof of a human or other animal. One or more sensing devices may include one or more electrode units, conductive sheets, sphygmomanometers, spirometer, acoustic blood pressuring devices, imaging units, capnography monitors, pulse oximeters, the like, or any combination thereof.

The one or more sensing devices may include one or more electrode units. The one or more electrode units may function to operate in a field-sensing mode, detect an electric field at a location in proximity with a subject's skin, detect and transmit one or more biopotential signals, or any combination thereof. The one or more electrode units may function to detect an electric field at a location in proximity but distanced (e.g., not in contact with) from a subject's skin. The one or more electrode units may include one or more contact electrode units, contactless electrode units, or both. The one or more electrode units may employ one or more current sensing electrode units, field sensing electrode units, or a combination of current sensing and field sensing units. The one or more electrode units may include one or more sensor elements, resistive sensor elements, capacitive sensor elements, amplifiers, conductors, or a combination thereof. The present teachings may incorporate one or more electrode units, sensor elements, or portion thereof as described in U.S. Pat. No. 10,182,732, such as in col. 11, line 18 to col. 13, line 10 (and associated drawings), incorporated herein by reference. One or more electrode units may include a single or a plurality of electrode units. The electrode units may include one or more, two or more, or even three or more electrode units. The electrode units may include twelve or less, nine or less, or even six or less electrode units. The one or more electrode units maybe communicatively coupled to a base unit.

One or more sensor elements may include one or more resistive sensor elements. One or more resistive sensor elements may sense current through or voltage across the resistive sensor element. One or more resistive sensor elements may require being placed in direct contact with the skin of a patient. In such a case, the one or more outer layers may include one or more openings therethrough which align with the resistive sensor element. This exposure is such that the one or more resistive sensor elements are able to come into contact with the skin of a patient. Examples of resistive sensor elements are described in U.S. Pat. No. 10,182,732 at col. 14, line 57 to col. 15, line 42 (and associated drawings), incorporated by reference herein.

One or more sensor elements may include one or more capacitive sensor elements. One or more capacitive sensor elements may detect the presence of an electric field. One or more capacitive sensor elements may allow the electrode unit to operate in a field-sensing mode, avoid having to be in direct contact with the skin of a subject, and even be distanced from the skin of the subject. A capacitive sensor portion may include a proximate surface. The proximate surface may be the sensing surface. The proximate surface may face toward, be adjacent to, or both the one or more outer layers. The proximate surface may face toward the skin of the subject (e.g., backside). Examples are described in U.S. Pat. No. 10,182,732 at col. 18, line 40 to col. 20, line 5 (and associated drawings), incorporated by reference herein.

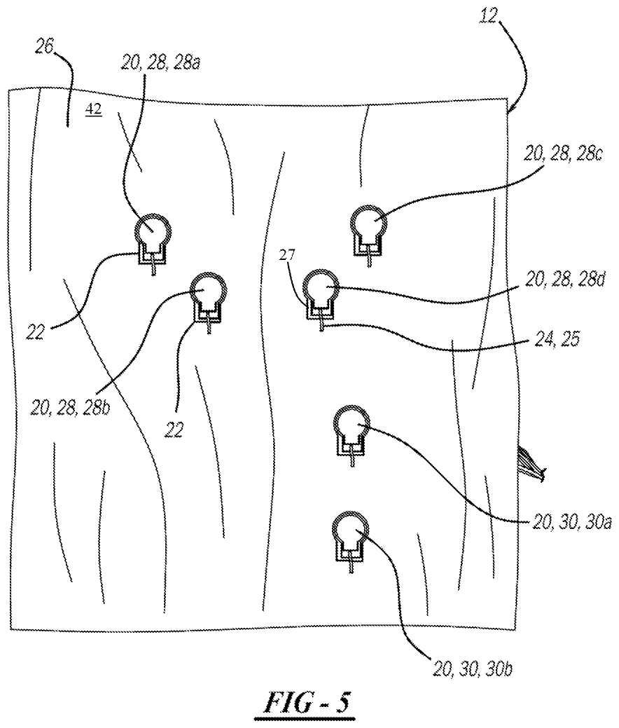

The one or more electrode units may include one or more contactless electrode units. The one or more contactless electrode units may function to detect an electric field at a location in proximity but distanced (e.g., not in contact with) from a subject's skin, detect biopotential signals from a subject without requiring skin contact, or both. One or more contactless electrode units may include one or more capacitive sensor elements. One or more suitable non-contact sensing devices are described as an electrode system is PCT Application No. PCT/US2019/063403 in par. no. [0037-0055] and FIGS. 4-5B (and any and all corresponding disclosure in U.S. Application No. 62/772,248 filed 28 Nov. 2018), and U.S. Pat. No. 10,182,732 at col. 12, line 18 to col. 14, line 56 and FIGS. 6A-6B, the teachings of each of which are incorporated herein by reference in its entirety. One or more suitable wired electrode units are described as an electrode system in PCT Application No. PCT/US2019/063403 in par. no. [0056-0058] and in FIG. 6A; incorporated herein by reference in its entirety. One or more suitable wireless electrode units are described as an electrode system in PCT Application No. PCT/US2019/063403 in par. no. [0059-0061] and FIG. 6B (and any and all corresponding disclosure in U.S. Application No. 62/772,248 filed 28 Nov. 2018), incorporated herein by reference in its entirety. A suitable example of a plurality of electrode units cooperating are described as an electrode system in PCT Application No. PCT/US2019/063403 in par. no. [0062-0066] and FIG. 6C (and any and all corresponding disclosure in U.S. Application No. 62/772,248 filed 28 Nov. 2018), incorporated herein by reference in its entirety.

The contactless electrode units may be able to detect biopotential signals through a plurality of layers, while a subject is clothed, or both. The contactless electrode unit may be able to detect a biopotential signal through the thickness of an outer layer of the support surface and a medical gown of a patient (e.g., 2 layers). The contactless electrode units may be able to detect a biopotential signal through the thickness of a permanent outer layer (e.g., PVC, leather, and the like), a temporary outer layer (e.g., poly-paper), a base layer of a subject (e.g., shirt), and even a subsequent layer (e.g., sweatshirt, sweater, sportscoat). The contactless electrode units may be able to detect a biopotential signal through the thickness of a permanent outer layer (e.g., PVC, leather, and the like), a temporary outer layer (e.g., poly-paper), a base layer of a subject (e.g., shirt), a mid-layer of the subject, (e.g., sweatshirt, sweater, sportscoat), and even an outer layer of the subject (e.g., jacket, vest, coat).

The one or more electrode units may include one or more amplifiers. One or more amplifiers may function to amplify the one or more signals recognized by one or more sensor elements, transmit one or more signals from the sensor elements, or both. Each of the plurality of electrode units may comprise a contact or contactless electrode and an amplifier circuit. Amplifier circuits may, for example, condition (e.g., amplify, filter, etc.) signals generated by or through electrodes respectively. Outputs of amplifier circuits may be communicatively coupled to a base unit for amplified signals to be transmitted to base unit. Amplified signals may be transmitted to base unit using a wired and/or wireless interface. One or more conductors may transmit the one or more amplified signals from the one or more amplifiers.

The one or more electrode units may be configured in a formation. The formation may allow for the electrode units to be located in proximity to common body portions of various subjects while remaining static within the support surface. The formation may allow for sensing elements of electrode units to be in proximity of a subject's torso, limbs, or both. The formation may allow for the sensing elements near the torso to be in close proximity to the origin of a limb and avoid needing to be placed at the ends of a limb. The origin of a limb may be the start of a limb from the torso (e.g., shoulder for arms, hip for legs). This formation allows for the support surface to only need to be sized such as accommodate torsos of individuals. The formation may include a plurality of electrode units arranged to form an outline of a shape. One or more electrode units may be arranged to form one or more vertices (e.g., corners), ends, or both of a shape. The shape may be one or more lines, triangles, squares, rectangles, circles, ovals, pentagons, trapezoids, diamonds, stars, the like, or a combination thereof. The shape may be right side up, upside down, or both. For example, a plurality of electrode units may form the vertices of an upside-down triangle. An upside down triangle may mean that a single vertices of the triangle may be closer and point toward the bottom face of the support surface while two vertices of the triangle may be closer to and point toward the top face and/or side faces. An upside-down triangle may place a single vertices closer to the hip of a subject with two vertices closer to the shoulders of a subject. The formation may include nested shapes. Nested shapes may include one or more inner shapes, middle shapes, outer shapes, or a combination thereof. Nested shapes may allow for the electrode units to remain static within the support surface while accommodating a large percentage of the population. Nesting allows for varying shapes of the sensing devices to accommodate varying anthropometry across the population. Nesting may allow for sensing devices associated with the same body portion and/or limp to cooperate together, compensate for each other, or both. For example, lower arm sensing devices may sense biopotential signals from a smaller female while upper arm sensing devices are not able to detect the signals. An inner shape may be an outline of a shape formed within an outline of another shape which is the outer shape. A middle shape may be an outline of a shape located between two outlines, the inner shape and outer shape. For example, the nested shapes may include an inner shape of an upside-down triangle formed by three electrode units located within an outer shape of an upside-down triangle formed by another three electrode units. The outer shape may include the most upper and lower electrode units. A formation of electrode units may be similar to that of Einthoven's triangle. The electrode units associated with each limb and/or corner may be aligned. Alignment may be vertical (e.g., parallel with a centerline), horizontal (e.g., perpendicular with the centerline), diagonal, the like, or any combination thereof.

The one or more electrode units may include one or more sensing devices associated with limbs. The one or more sensing devices may include arm sensing devices, leg sensing devices, or both. One or more arm sensing devices may include one or more upper arm, middle arm, lower arm, right arm, left arm, or a combination thereof (e.g., upper right arm) sensing devices. One or more leg sensing devices may include one or more upper leg, middle leg, lower leg, left leg, right leg, or a combination thereof (e.g., lower left leg) sensing devices. As an example, the one or more electrode units may include an upper right arm sensing device, lower right arm sensing device, upper left arm sensing device, lower left arm sensing device, upper leg sensing device, and lower leg sensing device. As another example, the one or more electrode units may include an upper right arm sensing device, middle right arm sensing device, lower right arm sensing device, upper left arm sensing device, middle left arm sensing device, lower left arm sensing device, upper left leg sensing device, middle left leg sensing device, and lower left leg sensing device. As another example, the electrode units may include a left arm sensing device and a right arm sensing device. The electrode units may include sensing devices configured to accommodate varying heights of subjects, such as a 5^{th} percentile female to a 95ₜₕ percentile male, such as an infant born prematurely at 21 weeks gestation to an infant at about 45 weeks gestation (i.e., infant born prematurely or timely plus time after birth), or a combination thereof.

The one or more electrode units may be distanced from one another, one or more resting surfaces, one or more faces, the like, or any combination thereof. The spacing may allow for the electrode units while statically affixed within the support surface to sense signals from individuals of significantly varying heights. For a support surface part of or used as a backrest (e.g., seated position of subject), distance measurements of electrode units may be measured relative to one another, a resting surface, and/or a face of the support surface. For a support surface part of or used as a lying mat (e.g., lying position of subject), distance measurements of electrode units may be measured relative to one another, a face of the support surface, or both. Distance measurements may be measured from center points of the sensing devices.

For a support surface useful as a backrest, for adults, or both, one or more upper arm sensing devices may be located below a sitting shoulder height of a 95′^{h} percentile male, 5^{th} percentile female, or both. One or more upper sensing devices may be located a height above a bottom face, resting surface, or both of about 25 cm or greater, about 30 cm or greater, about 35 cm or greater, or even about 40 cm or greater. One or more upper sensing devices may be located a height above a bottom face, resting surface, or both of about 65 cm or less, about 60 cm or less, about 55 cm or less, or even about 50 cm or less. For example, one or more upper sensing devices may be located about 35 cm to about 45 cm above a bottom face, resting surface, or both. One or more upper sensing devices may be offset from a centerline of a support surface. Offset may be horizontal, parallel with a top and/or bottom face, or both. One or more upper arm sensing devices may be distanced from a centerline by about 5 cm or greater, about 7 cm or greater, or even about 8 cm or greater. One or more upper sensing devices may be distanced from a centerline by about 15 cm or less, about 12 cm or less, or even about 10 cm or less.

For a support surface useful as a backrest, for adults, or both, one or more lower arm sensing devices may be located below the one or more upper arm sensing devices, diagonally offset from one or more upper arm sensing devices, or both. One or more lower arm sensing devices may be vertically below one or more upper arm sensing devices, closer to a centerline than one or more upper arm sensing devices, or both. One or more lower arm sensing devices may be located a height above a bottom face, resting surface, or both of about 20 cm or greater, about 25 cm or greater, about 30 cm or greater, or even about 35 cm or greater. One or more lower arm sensing devices may be located a height above a bottom face, resting surface, or both of about 60 cm or less, about 55 cm or less, about 50 cm or less, or even about 45 cm or less. For example, one or more lower arm sensing devices may be located about 30 cm to about 40 cm above a bottom face, resting surface, or both. One or more lower arm sensing devices may be offset from a centerline of a support surface. Offset may be horizontal, parallel with a top and/or bottom face, or both. One or more lower arm sensing devices may be distanced from a centerline by about 2 cm or greater, about 3 cm or greater, or even about 4 cm or greater. One or more upper sensing devices may be distanced from a centerline by about 12 cm or less, about 10 cm or less, about 8 cm or less, or even about 6 cm or less.

For a support surface useful as a backrest, for adults, or both, one or more upper leg sensing devices may be located below the one or more lower arm sensing devices, diagonally offset from one or more upper arm sensing devices, or both. One or more upper left leg sensing devices may be located a height above a bottom face, resting surface, or both of about 10 cm or greater, about 15 cm or greater, or even about 20 cm or greater. One or more upper left leg sensing devices may be located a height above a bottom face, resting surface, or both of about 35 cm or less, about 30 cm or less, or even about 25 cm or less. For example, one or more upper left leg sensing devices may be located about 20 cm to about 25 cm above a bottom face, resting surface, or both. One or more upper left leg sensing devices may be offset from a centerline of a support surface. Offset may be horizontal, parallel with a top and/or bottom face, or both. One or more upper left leg sensing devices may be distanced from a centerline by about 2 cm or greater, about 5 cm or greater, or even about 6 cm or greater. One or more upper sensing devices may be distanced from a centerline by about 15 cm or less, about 12 cm or less, about 10 cm or less, or even about 8 cm or less.

For a support surface useful as a backrest, for adults, or both, one or more lower leg sensing devices may be located one or more upper leg sensing devices, diagonally offset from one or more upper arm sensing devices, or both. One or more lower left leg sensing devices may be aligned with, offset, or both from the upper left leg sensing device. Alignment may refer to center-to-center being substantially parallel with centerline. One or more lower left leg sensing devices may be diagonally offset and distanced from one or more upper right arm sensing devices. One or more lower left leg sensing devices distanced from an upper right arm sensing device by about 10 cm or greater, about 12 cm or greater, about 15 cm or greater, or even about 17 cm or greater. One or more lower left leg sensing devices distanced from an upper right arm sensing device by about 30 cm or less, about 25 cm or less, about 23 cm or less, or even about 20 cm or less.

For a support surface useful as a horizontal mat, for infants, or both, one or more upper sensing devices may be distanced from one or more other upper sensing devices, lower sensing devices, or both. An upper left arm sensing device may be distanced from an upper right arm sensing device. The distance may be substantially perpendicular to a centerline of the support surface. The upper left arm sensing device may be distanced from the upper right arm sensing device by about 5 cm or greater, about 7 cm or greater, or even about 9 cm or greater. The upper left arm sensing device may be distanced from the upper right arm sensing device by about 15 cm or less, about 13 cm or less, or even about 11 cm or less. The upper left arm sensing device may be distanced from a lower left leg sensing device. The distance may be substantially parallel to a centerline of the support surface. The upper left arm sensing device may be distanced from a lower left leg sensing device by about 8 cm or greater, about 10 cm or greater, or even 11 cm or greater. The upper left arm sensing device may be distanced from a lower left leg sensing device by about 20 cm or less, about 15 cm or less, or even about 13 cm or less.

For a support surface useful as a horizontal mat, for infants, or both, one or more lower arm sensing devices may be distanced from one or more other lower arm sensing devices, leg sensing devices, or both. A lower left arm sensing device may be distanced from a lower right arm sensing device. The distance may be substantially perpendicular to a centerline of the support surface. The lower left arm sensing device may be distanced from the lower right arm sensing device by about 3 cm or greater, about 4 cm or greater, or even about 5 cm or greater. The lower left arm sensing device may be distanced from the lower right arm sensing device by about 10 cm or less, about 9 cm or less, about 8 cm or less, or even about 7 cm or less. The lower left arm sensing device may be distanced from an upper left leg sensing device. The distance may be substantially parallel to a centerline of the support surface. The lower left arm sensing device may be distanced from an upper left leg sensing device by about 3 cm or greater, about 4 cm or greater, or even 5 cm or greater. The lower left arm sensing device may be distanced from an upper left leg sensing device by about 10 cm or less, about 9 cm or less, about 8 cm or less, or even about 7 cm or less.

The system may include one or more conductive components. One or more conductive components may function to transmit one or more electrical signals within the system from one component to another component. One or more conductive components may include one or more conductive wires, cables, sheets, or any combination thereof. The one or more conductive components may be affixed to, in contact with, in electrical communication with, or a combination thereof one or more sensing devices, components thereof, a base unit, one or more signal converters, one or more computing platforms, or any combination thereof. For example, one or more conductive cables may include a 3-lead ECG trunk cable. The 3-lead ECG trunk cable may be affixed to the base unit and/or a signal converter (e.g., digital to analog) and to a computing device with a user interface.

One or more conductive sheets may be part of one or more inner layers, encapsulated by one or more outer layers, or both. One or more conductive sheets may function as a sensing device. One or more conductive sheets may function to detect common-mode voltages to which the subject's body, the other sensing devices (e.g., electrode units), and other conductive components (e.g., wires, cables) are exposed. One or more conductive sheets may aid in common-mode rejection. One or more conductive sheets may be located adjacent to one or more deflecting materials, outer layers, or both. One or more conductive sheets may be located between one or more inner layers and an outer layer at the front face of the surface support. One or more conductive sheets may have openings reciprocal with the one or more wells, sensing devices, or both. The openings in the conductive sheet may receive the one or more sensing devices therein. One or more conductive sheets may be similarly distanced from, not in contact with, or both the skin of the subject as one or more sensing devices. One or more conductive sheets may have one or more conductive wires, sensors, amplifiers, and/or the like affixed thereon. The one or more conductive sheets may be or contain an electrical circuit. The electrical circuit may be a driven right leg circuit (DRL). A driven right leg circuit may be beneficial in reducing interference. Exemplary interference may include electromagnetic interference, such as from electrical power lines. A driven right leg circuit may actively cancel the interference.

One or more conductive sheets may be sized such as to have one or more of the sensing devices therethrough. One or more conductive sheets may be sized such as to have a plurality (e.g., all) of the sensing devices therethrough and/or located therein. One or more conductive sheets may be sized to maximize proximity to a surface area of a backside (e.g., torso) of a subject, be able to be contained within the support system, or both. The one or more conductive sheets may have a width greater than a maximum width of sensing devices from one another (e.g., electrode units) within the support surface, a width equal to or less than a width of the support surface, or both. The one or more conductive sheets may have a height greater than a maximum height of sensing devices from one another (e.g., electrode units) within the support surface, a height equal to or less than a height of a support surface, or both.

The system may include one or more base units. The one or more base units may function to house one or more processors, memory storage devices, user interfaces, or any combination thereof; receive power and transmit to one or more components of the system; connect one or more sensing devices to one or more processors, memory storage devices, or both; convert signals (e.g., waveforms); or any combination thereof. A base unit may be referred to as a signal processor, signal processing unit (SPU), or both. The base unit may comprise and/or be affixed to one or more power supplies, input output (I/O) modules, processing modules, memory storage devices, signal converters, or a combination thereof. The base unit may include, be in communication with, be connectable to, or a combination thereof one or more computing devices. The processing module may include one or more processors. The processing module may comprise a combining module, an analog to digital converter and a digital signal processing module. An analog to digital converter may convert an incoming analog biopotential signal from one or more sensing devices to a digital biopotential signal. The analog to digital converter may transmit a digital signal to a digital signal processing module. A digital signal processing module may be used to suppress one or more signal distorting elements from the biopotential signal. The power supply may generate power for the one or more electrode systems. The I/O module may comprise one or more output devices for outputting data (e.g., a processed biopotential signal) to a user such as, for example, one or more displays, a printer or the like. I/O module may also comprise one or more user interfaces. I/O module may comprise a suitable network interface for communicating data (e.g., biopotential signal, processed biopotential signal) to and/or from base unit via a suitable network. Exemplary base units suitable with the sensing devices disclosed herein include the base units disclosed in PCT Publication No. WO 2020/112871 and U.S. Pat. No. 10,182,732, both incorporated herein by reference in their entirety. A single support surface may be in communication with a single base unit. A plurality of support surfaces may cooperate together with a single base unit. A base unit may be separate from or integrated into the support surface, secondary support surface, a computing device, or any combination thereof. For example, a base unit may be integrated into a hospital bed or an incubator. As another example, a base unit may be integrated into the electronics of a vehicle, such into the wire harness of a vehicle seat, the controls within the dash, engine compartment, or elsewhere in the vehicle, or a combination thereof. As another example, a base unit may be integrated into a monitoring device.

The system may include one or more signal converters. The one or more signal converters may function to convert one or more incoming analog signals to digital signals, digital signals to analog signals, or both. The one or more signal converters may convert a signal such that it is compatible with a receiving component. A receiving component may include a base unit, a computing device, or both. A signal converter may be located within, in advance of, or after a support surface, sensing devices, a base unit, a computing device, or a combination thereof. One or more signal converters may include one or more analog to digital converters, digital to analog converters, or both. One or more analog to digital converters may convert one or more detected biopotential signals from their analog form to digital form prior to being processed by a component of the base unit (e.g., digital signal processing module). One or more digital to analog converters may convert one or more processed biopotential signals to a native format prior to transmitting to a computing device. One or more digital to analog converters may be located within the base unit or after and prior to a computing device. For example, a digital to analog converter in electrical communication with and located after a base unit or within the base unit and in electrical communication with and prior to a cardiac monitor, electrocardiograph device, or both. A digital to analog converter may receive one or more processed biopotential signals (e.g., digitized) from a base unit, convert one or more processed biopotential signals to analog biopotential signals, transmit one or more analog biopotential signals to a computing device, such as a cardiac monitor, electrocardiograph device, or both.

The support surface, secondary support surface, or both may have, or be affixed to, one or more power supplies. A power supply may provide power to a base unit, one or more sensing devices, processors, storage mediums, user interfaces, computing devices, the like, or a combination thereof. The power supply may be battery, solar, affixed to incoming alternating or direct current, the like, or any combination thereof.

The system may include one or more processors. The one or more processors may function to analyze one or more signals and/or data from one or more sensing devices, memory storage devices, databases, user interfaces, or any combination thereof; convert one or more signals to data suitable for analysis and/or saving within a database; or a combination thereof. The one or more processors may be located within or in communication with a base unit, one or more sensing devices, one or more computing devices, one or more memory storage devices, one or more user interfaces, one or more support surfaces, or any combination thereof. One or more processors may include a single or a plurality of processors. One or more processors may be in communication with one or more other processors. The one or more processors may function to process data, execute one or more algorithms to analyze data, or both. Processing data may include receiving, transforming, outputting, executing, the like, or any combination thereof. One or more processors may be part of one or more hardware, software, systems, or any combination thereof. One or more hardware processors may include one or more central processing units, multi-core processors, front-end processors, the like, or any combination thereof. The one or more processors may be non-transient. The one or more processors may be referred to as one or more electronic processors. The one or more processors may convert data signals to data entries to be saved within one or more storage mediums. The one or more processors may access one or more algorithms to analyze one or more data entries and/or data signals. The one or more processors may access one or more algorithms saved within one or more memory storage mediums. The one or more processors may execute one or more methods for identifying the presence of one or more abnormal conditions in one or more biopotential signals of a subject; execute one or more methods for diagnosing an individual and detecting the presence of a medical condition; one or more methods for machine learning to determine one or more biomarkers indicative of a disease infection; or both. The one or more processors may execute the one or more methods via one or more algorithms stored within and accessible from one or more memory storage devices. One or more algorithms or may represent one or more nodes of an artificial neural network.

The system may include one or more memory storage devices (e.g., electronic memory storage device). The one or more memory storage devices may store data, databases, algorithms, or any combination therein. The one or more memory storage devices may include one or more hard drives (e.g., hard drive memory), chips (e.g., Random Access Memory “RAM)”), discs, flash drives, memory cards, the like, or any combination thereof. One or more discs may include one or more floppy diskettes, hard disk drives, optical data storage media including CD ROMs, DVDs, and the like. One or more chips may include ROMs, flash RAM, EPROMs, hardwired or preprogrammed chips, nanotechnology memory, or the like. The one or more memory storage devices may include one or more cloud-based storage devices. The data stored within one or more memory storage devices may be compressed, encrypted, or both. The one or more memory storage devices may be located within, part of, or in communication with a base unit, one or more sensing devices, computing devices, one or more processors, one or more user interfaces, one or more support surfaces, or any combination thereof. One or more memory storage devices may be referred to as one or more electronic memory storage devices. One or more memory storage devices may be non-transient. One or more memory storage mediums may store one or more data entries in a native format, foreign format, or both. One or more memory storage mediums may store data entries as objects, files, blocks, or a combination thereof. The one or more memory storage mediums may include one or more algorithms, rules, databases, data entries, the like, or any combination therefore stored therein. The one or more memory storage mediums may store data in the form of one or more databases.

One or more computing devices may include one or more databases. The one or more databases may function to receive, store, and/or allow for retrieval of one or more data entries. The data entries may be values associated with one or more detected signals, results from one or more algorithms, or both. The one or more databases may be located within one or more memory storage devices. The one or more databases may include any type of database able to store digital information. The digital information may be stored within one or more databases in any suitable form using any suitable database management system (DBMS). Exemplary storage forms include relational databases (e.g., SQL database, row-oriented, column-oriented), non-relational databases (e.g., NoSQL database), correlation databases, ordered/unordered flat files, structured files, the like, or any combination thereof. The one or more databases may store one or more classifications of data models. The one or more classifications may include column (e.g., wide column), document, key-value (e.g., key-value cache, key-value store), object, graph, multi-model, or any combination thereof. One or more databases may be located within or be part of hardware, software, or both. One or more databases may be stored on a same or different hardware and/or software as one or more other databases. One or more databases may be located in a same or different non-transient storage medium as one or more other databases. The one or more databases may be accessible by one or more processors to retrieve data entries for analysis via one or more algorithms. The database may be suitable for storing a plurality of records. A record may be one or more signals collected from a single individual during their respective test.

One or more computing devices may include one or more user interfaces. The one or more user interfaces may function to display information related to a user, receive user inputs related to a user account, display data to a user, or any combination thereof. The one or more user interfaces may function to receive a health status of an individual, display a health status of an individual, or both. The one or more user interfaces may be suitable for receiving data (e.g., a pre-recorded biopotential signal, particulars of a subject, etc.) from a user. The one or more user interfaces may include one or more graphic user interfaces (GUI). The one or more graphic interfaces may include one or more screens. The one or more screens may be a screen located directly on a base unit, separate from the base unit, another computing device, or any combination thereof. Exemplary computing devices (computing platforms) may include one or personal computing devices, monitoring devices, vehicle computing devices, and the like. One or more personal computing devices may include one or more desktop computers, laptops, mobile devices (e.g., tablet, mobile phone), the like, or any combination thereof. One or more computing devices may include one or more cardiac monitors, electrocardiograph devices, the like, or a combination thereof. One or more vehicle computing devices may include a vehicle's electronic control unit (ECU), electronic control module (ECM), onboard diagnostics (OBD), or any combination thereof. Exemplary monitoring devices may include Philips IntelliVue MX800 bedside patient monitor and Philips IntelliVue X2 portable patient monitor. One or more computing devices may include a mobile computing device, non-mobile computing device, or both. The one or more graphic interfaces may include and/or be in communication with one or more user input devices. The one or more user input devices may allow for receiving one or more inputs from a user. The one or more input devices may include one or more buttons, wheels, keyboards, switches, USB drives, the like, or any combination thereof. The one or more input devices may be integrated with a graphic user interface, separate from, in communication with, or a combination thereof. For example, one or more input devices may include one or more touch-sensitive monitor screens. The one or more user interfaces may be part of one or more computing devices with one or more ports. The one or more ports may allow for the computing device to be in electrical communication with the system, base unit, sensing devices, support surface, or any combination thereof. The one or more signals from a support surface, one or more sensing devices, base unit, or combination thereof may be accepted by a computing device in analog form, digital form, may need to be converted to analog form, converted to digital form, or a combination thereof.

The system may include on or more algorithms stored therein. The one or more algorithms may be stored within one or more memory storage devices. For example, one or more algorithms may be stored within one or more storage mediums of a base unit or another computing device. The one or more algorithms may function to analyze one or more signals, convert one or more signals to data, save data within one or more databases, execute one or more methods, or any combination thereof. The one or more algorithms may include one or more methods for diagnosing an individual and detecting the presence of a medical condition; one or more methods for machine learning to determine one or more biomarkers indicative of a medical condition; or both. The one or more algorithms may be formatted as one or more sets of computer-readable instructions. The one or more algorithms may be accessible by one or more processors for their execution.

The teachings herein incorporate by reference the teachings of each of Patent Cooperation Treaty Patent Application Nos. PCT/US2019/063403 (Filed 26 Nov. 2019 for Contactless Electrode For Sensing Physiological Electrical Activity) and PCT/US2019/063410 (Filed 26 Nov. 2019 for Systems and Methods for Digitally Processing Biopotential Signals); U.S. Application No. 62/772,248 filed 28 Nov. 2018; and U.S. Pat. Nos. 10,182,723 and 10,182,732; and the teachings of each such patent filing are incorporated by reference herein for all purposes.

Biomarkers & Medical Condition Detection

The system of the present teachings may be particularly useful in detecting one or more biopotentials, vital signs, and/or other physiological signs. One or more of these biopotentials, vital signs, and/or physiological signs may function to identify one or more biomarkers. The one or more biomarkers may identify the presence of medical condition, even before the onset of one or more physiological symptoms of the medical condition. A medical condition may include a viral infection or other infection, illness, or other medical disorder or condition. An infection may include one or more viral infections, bacterial infections, or both. One or more viruses causing one or more viral infections may include Ebola, Marburg, HIV, influenza, rotavirus, SARS-CoV, SARS-CoV-2 (COVID-19), MERS-CoV, the like, or any combination thereof. A medical disorder or condition may include glucose events, such as low and/or high blood glucose levels (e.g., hypoglycemia, hyperglycemia), stroke, heart attack, seizure, cardiac arrythmia, high and/or low blood pressure, fainting, falling asleep, inebriation, abnormal breathing rate, anxiety, the like, or any combination thereof. A medical condition may be identifiable by one or more biomarkers derived from one or more signals detected by one or more sensing devices. Each medical condition may be identified by a unique biomarker, combination of biomarkers, or both. The unique biomarker or combination of biomarkers may be determined via one or more machine learning algorithms. The presence of a unique biomarker or combination of biomarkers and the identification of one or more medical conditions may be determined by one or more detection algorithms.

One or more biopotentials may be identifiable by an electrocardiograph device. These one or more biopotentials may be represented by one or more waveforms. The one or more waveforms may include and/or identify one or more waves, intervals, segments, the like, or any combination thereof. The one or more waves may include a P wave, Q wave, R wave, T wave, S wave, U wave, J wave, Delta wave, Epsilon wave, or any combination thereof. The one or more intervals and/or segments may include a PR interval, PR segment, QT interval, ST segment, J point, QRS complex, the like, or any combination thereof. One or more waves may define an area, such as an area under the wave. The system of the present teachings may be particularly useful in characterizing the waveform morphology of the one or more waveforms and identifying one or more biomarkers by a phenomenon in the one or more waveforms.

The one or more biopotentials identifiable by an electrocardiograph device may identify one or more biomarkers. These biomarkers may be referred to as “electrocardiographic biomarkers.” The electrocardiographic biomarkers may function to identify the presence of a medical condition, even before the presence of other physiological symptoms. The one or more biomarkers may include: QTc interval prolongation, T wave depression, R wave depression, QRS prolongation, ST segment elevation, ST segment depression, R wave depression, R to R interval, one or more rhythm disturbances, one or more conduction defects, the like, or any combination thereof. One or more rhythm disturbances may include ventricular premature complexes, supraventricular premature complexes, sinus arrhythmia, sinus tachycardia, the like, or any combination thereof. One or more conduction defects may include 1 degree AV block, 2 degree AV block, right bundle branch block, the like, or any combination thereof. For example, a QTc interval prolongation and T-wave depression may be indicative of the presence of COVID-19 before any other physiological symptoms are present.

One or more vital signs, other physiological signs, or both may function to cooperate with the one or more biopotentials to identify the presence or absence of one or more biomarkers. The one or more vital signs may include a pulse rate, temperature, respiration rate, blood pressure, the like, or any combination thereof. The one or more vital signs may be indicative of an individual's essential body functions. One or more vital signs may be determined by one or more sensing devices. One or more vital signs may be determined using the same sensing devices as those detecting the one or more biopotentials or other sensing devices. For example, pulse rate can be determined using one or more electrode units in the support surface. As another example, a respiration rate may be estimated based on one or more algorithms applied to the biopotential signals from the electrode units, a capnography monitor, a spirometer, or a combination thereof. The one or more physiological signs may include peripheral blood flow, peripheral capillary oxygen saturation, sweat rate, skin conductance, skin temperature, headache, coughing, one or more gastrointestinal issues, muscle weakness, the like, or any combination thereof. One or more physiological signs may include one or more conditions detected by one or more imaging methods. One or more imaging methods may include ultrasonic imaging, computer tomography imaging, x-ray imaging, magnetic resonance imaging, the like, or any combination thereof. The one or more vital signs, physiological signs, or both may cooperate with one or more electrocardiographic biomarkers to detect and/or identify the presence of one or more infections or diseases. As an example, a slightly elevated temperature (e.g., between 99° F. and 100° F.) in combination with a QTc interval prolongation and T wave depression may be indicative of the presence of COVID-19 before other physiological symptoms are present.

Method for Diagnosing an Individual and Detecting Presence of a Medical Condition

The present disclosure relates to a method for diagnosing individuals to detect, and/or monitor progression of, the presence and/or absence of one or more medical conditions. One or more medical conditions may include infection, illness, or other medical disorder or condition. For example, the presence of a virus. The method may use the system according to the teachings herein. The method may allow for the collection of data of individuals. The method may be at least partially automatically performed by one or more processors of a computing device part of or separate from a base unit, monitoring device, personal computing device, or combination thereof. The method may include providing a support surface, positioning the human on the support surface, acquiring data from one or more sensing devices, outputting the data, analyzing the data, or a combination thereof. The method may be performed before a human has the onset of any physiological symptoms, after birth, during regular lifestyle activities such as driving or sleeping, while under medical care, or any combination thereof. The method (or steps involving data acquisition) may be performed 2 or more, 4 or more, 8 or more, 12 hours or more, 18 hours or more, 24 hours or more, 36 hours or more, or even 48 or 72 hours or more before a subject (e.g., human) has the onset of any physiological symptoms associated with a medical condition. Thus, the method (or steps involving data acquisition) may be employed prior to (e.g., 2 or more, 4 or more, 8 or more, 12 or more, 18 or more, 24 or more, 36 or more, or even 48 or 72 hours or more before) the onset of objectively detectable symptoms (e.g., before a patient temperature reading departs (such as by at least 0.5, 1, 1.5 or 2° C.) from a typical normal temperature of 37° C.), and/or other identified symptoms (e.g., sore throat, persistent cough, difficulty breathing, pain, etc.).

The method for testing an individual may include providing a support surface. The support surface has one or more sensing devices embedded therein, connected thereto, in proximity, or a combination thereof. The support surface may be integrated into a secondary support surface, placed on a secondary support surface, or both. For example, a support surface may be located onto a base (e.g., resting surface) of an incubator configured for infants. As another example, a support surface may be placed on a backrest of a chair. As a further example, the support surface may be built into backrest of a seat of vehicle, a mat and/or pad of a bed, a backrest of a chair, or the like. The method may include powering the support surface. This may include placing one or more conductors from the support surface in electrical communication with a base unit, power supply, or both. The method may include placing the support surface in electrical communication with one or more computing devices. This may include placing a base unit in electrical communication with a computing device, the support surface in electrical communication with a base unit, the support surface in electrical communication with computing device, or any combination thereof.

The method may include placing one or more outer layers on a support surface. The one or more outer layers may be placed prior to and/or after placement of a support surface on a secondary support surface, during integration of the support surface into a secondary support surface, or a combination thereof. An outer layer may be placed over an inner layer and the sensing devices prior to placement of the support surface on a secondary support surface. As another example, a temporary outer layer, such as poly-paper, may be placed over a permanent outer layer after the support surface is located on a secondary support surface.

The method may include positioning a subject, at least partially, on the support surface. This positioning may allow for one or more sensing devices to detect and collect one or more signals from the subject (e.g., human). The subject may be a human. The subject may range from infant to adult. The subject may be a patient, medical worker, hospital worker, resident, driver, passenger, the like, or any combination thereof. The subject may be placed such that their back is placed against a front surface of the support surface. The subject may be placed such that the tops of their shoulders and/or width of their shoulders are located within the boundary (e.g., periphery) of the support surface. The subject may be placed such that their hips are located within the boundary of the support surface. The subject may be placed such that just their torso is in contact within the boundary of the support surface. The subject may be places such that an entire length of their body (e.g., height) is within the boundary of the support surface. For example, adults may be positioned on a support surface such that just the backside of their torso is against the support surface. For example, infants may be positioned on a support surface such that a backside of the entire length of their body against the support surface. The one or more signals may be one or more biopotentials, vital signs, other physiological signs, or a combination thereof. The subject (e.g., human) may be placed on an outer layer of a support surface. The subject may be placed against a front face of a support surface. The subject (e.g., human) may be placed in direct and/or indirect contact with one or more sensing devices. The subject (e.g., human) may be placed in proximity and/or adjacent to one or more sensing devices.

The method may include acquiring data from the system. Data may also be input by an individual via a user interface. The data may be related to and/or represent the one or more signals detected and collected by the one or more sensing devices. The one or more signals may be transmitted to one or more processors, memory storage devices, or both. The data may be acquired in a continuous manner, periodic intervals, or both. Periodic intervals must be small enough to detect changes in morphology of a waveform related to one or more biopotentials. Data may also include additional data inputted from an individual. The additional data may include name, age, gender, race, ethnicity, comorbidity, income, career, education, the like, or any combination thereof of the subject (e.g., human).

The method may include outputting the data. Data may be automatically outputted. After the data is acquired, the data may be transmitted to one or more processors, memory storage devices, user interfaces, or any combination thereof. The data may be transmitted to one or more databases within one or more memory storage devices. The data may be transmitted to one or more base units, components of a base unit, computing devices, or any combination thereof.

The method may include analyzing the data. Analyzing may be completely or partially automatic by one or more processors. Analyzing of the data may include analyzing a waveform, value, or both of one or more biopotentials, vital signs, and/or other physiological signs. Analyzing may include identifying one or more biomarkers consistent with a medical condition. Analyzing may include a step of comparing at least a portion of the collected data with pre-existing biomarker data. The pre-existing biomarker data may be data indicative of a medical condition. The pre-existing biomarker data may be determined via a machine learning process. The pre-existing biomarker data may include one or more data values for one or more vital signs, physiological signs, or both. The pre-existing biomarker data may include one or more data values for one or more biopotential waveforms. The pre-existing biomarker data may include morphology regarding one or more biopotential waveforms. For example, an amplitude, volume under a curve, segments, depressions, prolongations, the like, or any combination thereof. A certain morphology of a waveform may indicate the presence of a certain disease condition. The pre-existing biomarker data may be indicative of a virus. Analyzing the data may be completed by one or more processors.

The method may include repeating acquiring the data, outputting the data, analyzing the data, or a combination thereof. Repeating may occur for a same subject (e.g., human), one or more different subjects (e.g., humans). Repeating may occur within the same day or separate days. Separate days may or may not be consecutive.

Any of the teachings described herein may employ methods and/or an apparatus for digitally processing one or more acquired biopotential signals. For example, in accordance with the teachings herein related to a support system, medical conditions, and biopotential signals; and in accordance with the teachings of paragraphs [0049]-[0153] of Patent Application No. PCT/US2019/063410 (and any and all corresponding disclosure in U.S. Application No. 62/772,248 filed 28 Nov. 2018), all incorporated by reference herein.

Any of the teachings described herein may employ a methods and/or apparatus for suppressing one or more signal distorting elements from an acquired biopotential signal, e.g., in accordance with the teachings of paragraphs [0043]-[0052] of Patent Application No. PCT/US2019/063410 (and any and all corresponding disclosure in U.S. Application No. 62/772,248 filed 28 Nov. 2018), all incorporated by reference herein. For example, one or more steps may be performed to suppress one or more motion artefacts present in an acquired biopotential signal (i.e., suppress signal distortions introduced into biopotential signal as a result of movement of a subject during acquisition of biopotential signal). This may involve digitally processing a biopotential signal (e.g., by filtering, by using a moving average technique (such as Zero Lag Exponential Moving Average), or both, to suppress artefacts.

Any of the teachings described herein may employ methods and/or apparatus for processing one or more acquired biopotential signals using Empirical Mode Decomposition. An example of this may be in accordance with the teachings of paragraphs [0053]-[0074] and associated drawings of Patent Application No. PCT/US2019/063410 (and any and all corresponding disclosure in U.S. Application No. 62/772,248 filed 28 Nov. 2018), all incorporated by reference herein. Empirical Mode Decomposition (EMD) may include decomposing a biopotential signal into a plurality of “Intrinsic Mode Functions” (IMFs), where the sum of the IMFs reconstruct decomposed biopotential signal. In particular, EMD may iteratively parse biopotential signal into a plurality of “fast oscillation” and “slow oscillation” components (each component corresponding to a different IMF). Upon biopotential signal being parsed into a plurality of IMFs, one or more IMFs corresponding to (e.g. comprising or otherwise corresponding to) artefacts and/or noise may be identified. Identified artefacts and/or noise may, for example, be suppressed by reconstructing biopotential signal using only IMFs not identified as corresponding to artefacts and/or noise (i.e. any IMFs identified as corresponding to artefacts and/or noise are excluded during reconstruction of the biopotential signal).

Any of the teachings described herein may employ methods and/or apparatus for processing one or more acquired biopotential signals using a Wavelet transform. An example of this may be in accordance with the teachings of paragraphs [0075]-[0090] of Patent Application No. PCT/US2019/063410 (and any and all corresponding disclosure in U.S. Application No. 62/772,248 filed 28 Nov. 2018), all incorporated by reference herein.

Any of the teachings described herein may employ methods and/or apparatus for processing one or more acquired biopotential signals using an Independent Component Analysis. An example is in accordance with the teachings of paragraphs [0091]-[0102] and associated drawings of Patent Application No. PCT/US2019/063410 (and any and all corresponding disclosure in U.S. Application No. 62/772,248 filed 28 Nov. 2018), all incorporated by reference herein. An example method of ICA may take several input signals (each signal comprising a plurality of sources) and may extract each of the plurality of sources from each signal. In embodiments where the plurality of biopotential signals comprises, for example, ECG signals generated using a plurality of electrodes placed at different locations on a subject's body, each biopotential signal may comprise a plurality of sources such as, for example, ECG data, noise (e.g., 60 Hz electromagnetic interference) and/or artefacts (e.g. motion artefacts, etc.). Each biopotential signal may comprise the same and/or different sources compared to the other biopotential signals in the plurality of biopotential signals. A method of ICA may, for example, extract ECG data while suppressing other sources (e.g., noise sources, artefact sources, etc.).

Any of the embodiments described herein may employ methods and/or apparatus for extrapolating physiological parameters from noisy biopotential signals. See, Patent Application No. PCT/US2019/063410 (and any and all corresponding disclosure in U.S. Application No. 62/772,248 filed 28 Nov. 2018), all incorporated by reference herein.

Machine Learning to Determine Biomarkers Indicative of Medical Condition

The present disclosure relates to a method of machine learning to determine one or more biomarkers indicative of a specific medical condition. The method of machine learning may include providing a support surface, placing one or more outer layers on or as part of the support surface, positioning the subject (e.g., human) on the support surface, acquiring data from one or more sensing devices, outputting the data, analyzing the data, labeling the data, or a combination thereof. The method may be performed on a subject (e.g., human) who is healthy, has a virus or other disease or medical condition, or both. The method may be initially performed on a subject (e.g., human) when they are healthy, such as to establish a baseline, and then later when they have a confirmed medical condition, such as a virus or other disease. The method may be performed by one or more processors of one or more computing devices. The method may be automatically performed by one or more processors of a computing device part of or separate from a base unit, monitoring device, personal computing device, or combination thereof. The method may allow for the collection of data from a number of subjects useful for determining biomarkers related to a variety of medical conditions. The method may allow for collection of data not only from medical patients in medical settings and determining biomarkers indicative of medical conditions, but also within lifestyle settings, such as in a vehicle while driving.

The method of machine learning may include a neural network (e.g., “artificial neural network”). Neural networks are generally presented as systems of “neurons” which can compute values from inputs, and as a result of their adaptive nature, are capable of learning and/or recognizing patterns. The neural network may be a deep neural network (DNN). A neural network may function my linking a plurality of nodes. The plurality of nodes may be within one or more input layers, hidden layers, output layers, or a combination thereof. The one or more input layers may be associated with each individual biopotential signal, vital signal, and/or physiological signal collected by one or more sensing devices. The one or more output layers may be a determination of the presence and/or absence of a specific medical condition. Each node may be responsible for a computation (e.g., execution of an algorithm). Exemplary neural networks, configurations, and training methods are discussed in “Deep Learning” by Ian Goodfellow, et al, Massachusetts Institute of Technology, 2016; incorporated herein by reference in its entirety.

One or more of the steps of providing a support surface, placing one or more outer layers, positioning the subject (e.g., human) on the support surface, acquiring data, outputting the data, and/or analyzing the data may be the same or similar as those for the method for testing individuals to detect the presence and/or absence of medical condition.

The method of machine learning may include a method of training. Training may allow for the machine learning method to learn how to automatically identify one or more biomarkers indicative of one or more medical conditions, save the one or more biomarkers as pre-existing biomarker data, identify one or more medical conditions by comparing to the pre-existing biomarker data, or any combination thereof. Training may include supervised learning, unsupervised learning, reinforcement learning, or any combination thereof. The more data that is collected and analyzed, the more accurate the detection of one or more medical conditions, such as infections and/or diseases, may be. The training method may be completed in a live mode, offline mode, or both. The live mode may collect data from one or more subjects (e.g., humans) and simultaneously capture their health status. The health status may be input by the user via a user interface. For example, a number of individuals may be asked to sit on a support surface (e.g., chair) equipped with one or more sensing devices. The system detects and collects one or more biopotential signals, vital signals, physiological signals, or any combination thereof and stores these signals as data within a memory storage device. The user inputs their health status via a user interface. Their health status may be determined from a prior clinical test and result (e.g., swab test, medical diagnosis). Health status may indicate healthy, presence of a medical condition, or both. Presence of a medical condition may include presence of a condition, infected, infected with a specific infection, or a combination thereof. The health status of an individual is automatically appended as a data label to the data collected by the system. The health status is appended as a data label by a processor. The offline mode may append health status data to previously recorded data. For example, a number of individuals may have previously been asked to previously sit on a support surface and have one or more biopotential signals, vital signals, and/or physiological signals collected. These individuals may have also already received results from a prior clinical test or other method of medical diagnosis. The health status of each individual may be associated with their previously recorded data as a data label. After data collection in live mode, offline mode, or both, the data of a plurality of individuals is stored in one or more databases with associated data labels indicative of their health status. Overtime, one or more records associated with one or more individuals may be removed from the system to save storage space, expedite processing, and the like. During training, a portion of the records must remain stored within the database. A portion of the records may include 5% or greater, 10% or greater, or even 20% or greater. A portion of the records may include 100% or less, 50% or less, or even 30% or less. For example, a portion of the records may be 20% to 30% of the records.

The method of machine learning may include a method of feature extraction. Feature extraction may function to extract useful data from the one or more signals detected by the one or more sensing devices. Future extraction may be performed automatically by one or more processors of a computing device. Feature extraction may include filtering the one or more signals for specific features of one or more biopotential signals, vital signals, physiological signals, or any combination thereof (e.g., “incoming signals”). Feature extraction may include filtering the one or more incoming signals for data useful by one or more nodes of a neural network. Feature extraction may include collecting values in a certain format associated with the incoming signals. The values may be parametric values. Feature extraction may include collecting values associated with the waveform morphology associated with one or more biopotential signals. Once extracted, the features may be saved in one or more databases, associated with a record, or both. After feature extraction, a data label may be associated with each record.

The method of machine learning may include a method of extracting time series data. Time series data may cooperate with or be used in lieu of feature extraction data. Time series data may allow for an entire morphology of a waveform from one or more incoming signals to be analyzed as opposed to certain features (values) at certain times of a waveform. Extracting time series data may be performed automatically by one or more processors of one or more computing devices. Extracting a time series may involve breaking down one or more incoming signals into one or more frames, one or more frames into one or more subsamples, saving data associated with the one or more frames and/or subsamples, or any combination thereof. One or more frames may be over a certain period of time. The period of time may be about 5 seconds or more, 10 seconds or more, 20 seconds or more, or even 30 seconds or more. The period of time may be about 10 minutes or less, about 5 minutes or less, about 2 minutes or less, or even 1 minute or less. For example, the method of extracting time series data for an incoming bipotential signal begins with R-R interval detection of a biopotential waveform. An R-R interval may be the time elapsed between two successive R-waves of a QRS signal. A typical R-R interval may be anywhere from about 0.5 to about 1.2 seconds. After R-R interval detection, resampling is applied to fit around each detected R-R frame. Resampling may mean that one or more frames may be broken down into a number of samples (e.g., subsamples). A frame may be broken down into about 50 or more samples, about 100 or more samples, about 120 or more samples, or even about 128 or more samples. A frame may be broken down into about 2048 or less, 1024 or less, 512 or less, or less samples, about 500 or less samples, about 300 or less samples, or even about 256 samples. Each sample may be associated with one or more values of the incoming signal at the respective interval to which the frame is broken down. By ensuring an individual R-R frame is detected, the process may ensure there is always a full R-R cycle available for analysis of the waveform morphology. An initial frame may be predetermined. The frame size (e.g., number of samples in a frame) may be adjusted to allow for a balance between detection quality and processing power requirements. Time series may require more nodes within a neural network as compared to feature extraction but may also provide additional accuracy.

The method of machine learning may include a method of data labeling. Data labeling may function to associate one or more records with one or more health status; to allow for machine learning to be trained to identify one or more health status by one or more values saved within a record, or both. Data labeling during machine learning may allow for the one or more data labels to be used as one or more health status outputs after the training phase, during the diagnosis method, or both. Data labeling may be performed manually by an individual, automatically by a processor of a computing device, or both. Data labeling may be input in a live mode, offline mode, or both. Data labeling may include associating a health status to a record. A health status may include presence of a medical condition, healthy, infected, infected with a certain infection and/or disease, or a combination thereof. For example, health statuses may include healthy, COVID-19 (e.g., identifying an individual that has tested positive for COVID-19), and other (e.g., identifying an individual that has tested positive for another infectious disease). Data labeling may change and/or be updated for a record. For example, individuals may be taking clinical tests on regular intervals. When an individual had their individual data collected by the system, the individual had recently received a healthy diagnosis. Thus, the data label associated with their record was initially healthy. A day later, the individual received an updated clinical test result, indicating they are infected with COVID-19. If there is a chance the individual was already infected when they underwent data collection by the system, the data label associated with the data record may be updated to reflect they were infected with COVID-19. The regular updating of records may allow for the training method to more quickly determine one or more biomarkers associated with an infection and/or disease. Data labeling may be necessary for the machine learning to calculate weights during a back propagation and/or training process. By knowing a desired output in a form of a data label, the machine learning is able to calculate an error and back propagate proper weights to a plurality of nodes from the input layer to the output layer.

The method of machine learning may include analyzing the data to determine one or more morphologies indicative of a medical condition, such as a disease. Analyzing the data may be automatically performed by one or more processors of one or more computing devices. Analyzing the data may include correlating the collected data and the one or more data labels to determine the one or more biomarkers indicative of a disease condition. Analyzing the collected data may include automatically evaluating a waveform morphology. Evaluating a waveform morphology may include a predetermined number of amplitudes, samples, or both selected over a predetermined period of time. For example, the predetermined period of time may be an R-R frame. The amplitude readings may be compiled, correlated with a user, other bioinformation of the user, and stored in memory for subsequent retrieval and use. Readings for one or more users over time may be monitored to establish morphological changes over time for onset of a disease and/or after the onset of a disease.

Biopotentials as Identification of Individuals

The present disclosure relates to a method for determining the biopotential fingerprint of an individual. The method may include providing a support surface with one or more sensing devices of an electrocardiograph embedded therein and/or providing the one or more sensing devices of the electrocardiograph device; placing one or more outer layers as part of a support surface; positioning the subject at least partially on the support surface and/or positioning the one or more sensing devices on the subject; acquiring data from the electrocardiograph reading on the subject; outputting the data; correlating the data to the subject as a record. At least some of these steps may be similar and/or the same as those for methods of diagnosing a medical condition, machine learning for medical conditions, or both as described herein.

The outputted data may save certain values, parametric values, data series, the like, or a combination thereof related to one or more biopotential waveforms of an individual subject. Saving of data may be automatically completed by one or more processors. The one or more processors may be from a base unit, computing device, the like, or a combination thereof. The outputted data may serve as a unique digital fingerprint which identifies one or more subjects. The outputted data may serve as one or more inputs into a neural network. The outputted data may have one or more data labels affixed thereto. The one or more data labels may be identifiers of the subject. The one or more data labels may include the subject's name, residence, birthdate, race, ethnicity, gender, education, career, salary, the like, or any combination thereof.

After initial data collection, one or more subjects (same or different) may have their biopotential data collected. These one or more subjects may or may not have had their data previously collected. This subsequent data may be compared to all of the biopotential data stored within a database. Comparing may be automatically completed by one or more processors. The method may include filtering all of the biopotential data stored within the database for one or more substantially similar biopotential waveforms. The method may include using a neural network to analyze the morphology of one or more biopotential waveforms and comparing to the morphology of one or more saved waveforms. The individual may be identified even if their biopotential data has never been collected. For example, key traits of their biopotential wave morphology may identify the subject by age, gender, race, ethnicity, other demographic data, living conditions, etc. These traits may be compared against data in one or more other databases to filter for one or more subjects meeting similar criteria. Once at least one substantially similar biopotential waveform is identified and/or similar traits, the system may identify the subject by the data label associated with the similar record, by one or more traits in another database, the like, or a combination thereof.

### ILLUSTRATIVE EMBODIMENTS

Any of the features or processes described herein may be combined with one another in any combination. Any of the features or processes described herein may be combined or used in lieu of one or more features described in U.S. Pat. No. 10,182,732; US Patent Application Publication No.: 2019/0090747; and PCT Application Nos. PCT/US2019/063403, PCT/US2019/063410; and U.S. Application No. 62/772,248 filed 28 Nov. 2018), which are incorporated herein by reference in their entirety for all purposes.

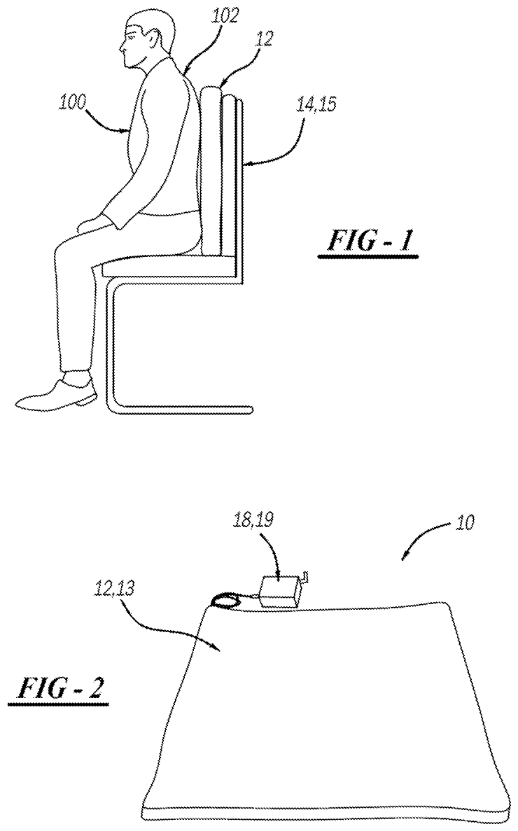

FIG. 1 illustrates a subject 100 positioned on a support surface 12. A back 102 of the subject 100 faces toward the support surface 12.

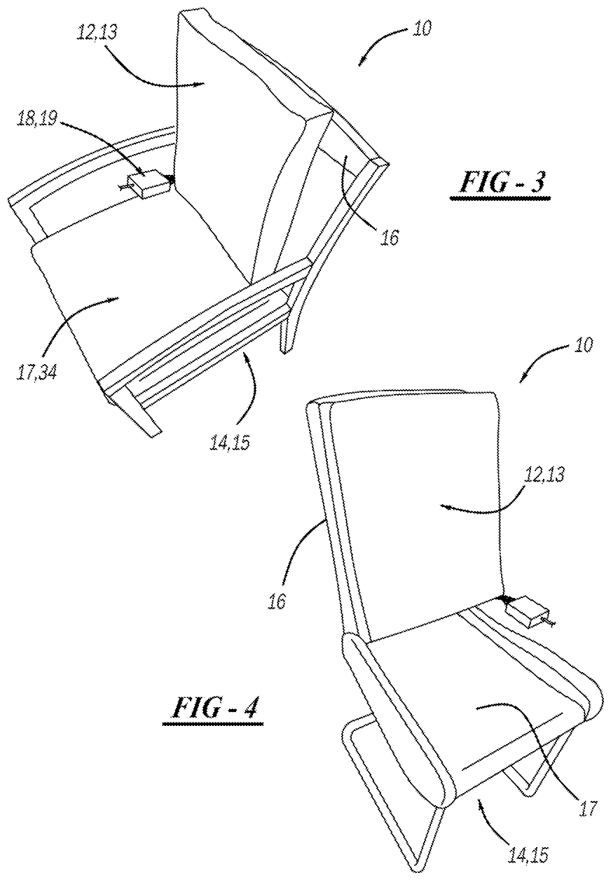

FIGS. 2 to 4 illustrate a system 10 according to the teachings herein. The system 10 includes a support surface 12. The support surface 12 includes a pad 13. The support surface 12 is configured to be supported by a secondary support surface 14. The secondary support surface 14 is illustrated as a chair 15. The pad 13 rests upon the backrest 16 of the chair 15. The pad 16 is thus able to be located between the back of a subject and the backrest. The support surface 12 is located between arms of the secondary support surface 14. The pad 13 rests on the seat cushion 17. The seat cushion 17 acts as a resting surface 34. The system 10 includes a signal processor 18. The signal processor 18 may be a signal processing unit (SPU) 19. The support surface 12 includes one or more sensing devices 20 (not shown). The one or more sensing devices 20, a part of the support surface 12, or both are in electrical connection with a signal processor 18. The electrical connection(s) may be similar to those illustrated in FIG. 6.

FIG. 5 illustrates a plurality of sensing devices 20 part of a support surface 12. The support surface 12 includes one or more inner layers 26. The sensing devices 20 are located within openings 21 and wells 22. The openings 21 are aligned with the wells 22. The openings 21 and wells 22 are formed in the one or more inner layers 26. The openings 21 are formed in a conductive sheet 42. The wells 22 are formed in a deflecting layer 27. The conductive sheet 42 is located on the deflecting layer 27. The openings 21 and wells 22 are substantially reciprocal in shape with the sensing device 20. Each sensing device 20 is affixed to a conductor 24. The conductor 24 is in the form of an electrical cable 25. The conductor 24 is partially embedded within the inner layer 26. The sensing devices 20 include arm sensing devices 28 and leg sensing devices 30. The arm sensing devices 28 include an upper right arm sensing device 28a, lower right arm sensing device 28b, upper left arm sensing device 28c, and lower left arm sensing device 28d. The leg sensing devices 30 include an upper leg sensing device 30a and a lower leg sensing device 30b.

FIG. 6 illustrates a schematic of a plurality of sensing devices 20 of a support surface 12. The upper right arm sensing device 28a is distanced from the upper left arm sensing device 28c by a width W1. The upper right arm sensing device 28a is distanced from the centerline CL by a width W2. The lower right arm sensing device 28b is distanced from the centerline CL by a width W3. The upper leg sensing device 30a and lower leg sensing device 30b are distanced from the centerline CL by a width W4. The support surface 12 includes a bottom face 32. The bottom face 32 may abut to a resting surface 34 when positioned for use by a subject. The upper right arm sensing device 28a is distanced from the bottom face 32 by a height H1. The lower right arm sensing device 28b is distanced from the bottom face 32 by a height H2. The upper leg sensing device 30a is distanced from the bottom face 32 by a height H3. The lower leg sensing device 30b is distanced from the bottom face 32 by a height H4. The upper right arm sensing device 28a is distanced from the lower leg sensing device 30b by a first distance D1.

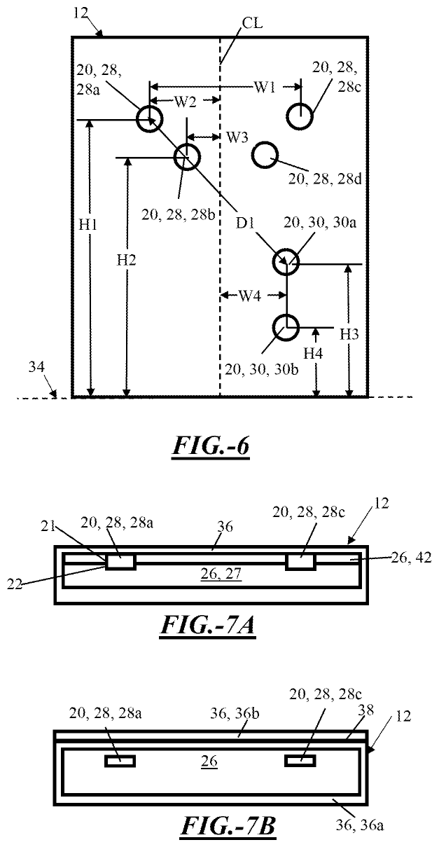

FIG. 7A illustrates a cross-section of a support surface 12 taken through sensing devices 20, such as the upper right arm sensing device 28a and upper left arm sensing device 28c. The sensing devices 20 are not completely covered by the one or more inner layers 26. The sensing devices 20 are directly adjacent to an outer layer 36. The outer layer 36 encapsulates about an entirety of a plurality of inner layers 26. The inner layers 26 include a deflecting layer 27 (e.g., foam) and a conductive sheet 42. The conductive sheet 42 includes a plurality of openings 21 through which the sensing devices 20 pass through. The deflecting layer 27 includes a plurality of wells 22 in which sensing devices 20 are located therein.

FIG. 7B illustrates a cross-section of a support surface 12 taken through sensing devices 20, such as the upper right arm sensing device 28a and upper left arm sensing device 28c. The sensing devices 20 are completely embedded within the one or more inner layers 26. The sensing devices 20 are distanced from the outer layer 36. The outer layer 36 includes a first outer layer 36a and a second outer layer 36b. The first outer layer 36a encapsulates about the inner layer 26. The second outer layer 36b extends over or is part of the first face 38. The first face 38 is the face of the support 12 which is closest to the back 102 (not shown) of a subject 100 (not shown). Any combination of features of FIG. 7A may be combined with FIG. 7B. For example, a conductive sheet could be employed in the example of FIG. 7B. As another example, a second outer layer 36b could be employed in the example of FIG. 7A.

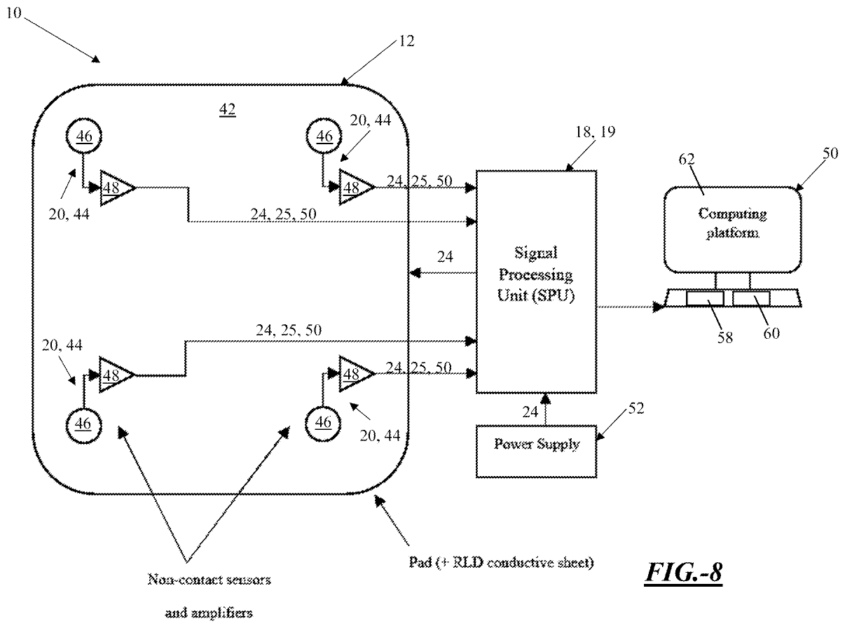

FIG. 8 illustrates a system 10. The system 10 includes a support surface 12. The support surface includes a conductive sheet 42. The conductive sheet 42 may be a driven right leg circuit (DRL). The DRL circuitry may allow for eliminating or cancelling out interference noise. The support surface 12 includes a plurality of sensing devices 20. Each sensing device 20 is an electrode unit 44. Each electrode unit 44 includes a non-contact sensor 46 and amplifier 48. Each sensing device 20 is in electrical communication with a signal processor 18. The signal processor 18 may be in the form of a signal processing unit (SPU) 19. The sensing devices 20 are connected to the signal processor 18 via conductors 24. The conductors 24 are electrical cables 25. The conductors 24 are individual analog cables 50 (i.e., one analog cable from each amplifier to the SPU). The signal processor 18 is also connected to the conductive sheet 42 via a conductor 24. The signal processor 18 may be part of or separate from a base unit. The signal processor 18 is powered from a power supply 52. The power supply 52 is connected to the signal processor 52 via a conductor 24. The conductor 24 is a power cable. The signal processor 18 is in communication with a computing platform 54. The signal processor 18 is in communication with the computing platform 54 wirelessly or via a digital cable 56. The computing platform 54 includes one or more processors 58, memory storage devices 60, and user interfaces 62. The computing platform may also be part of or separate from a base unit.

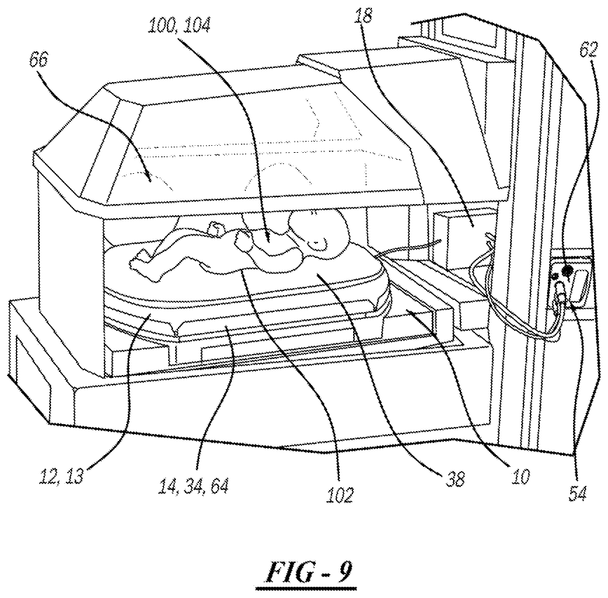

FIG. 9 illustrates a system 10. The system 10 includes a support surface 12. The support surface 12 includes a pad 13. The support surface 12 is supported by a secondary support surface 14. The secondary support surface 14 is a resting surface 34. The resting surface 34 is a supportive base 64 of an incubator 66. Located on the support surface 12 is a subject 100. The subject 100 is an infant 104. The subject's 100 back 102 rests on the support surface. The subject's 100 back 102 is located on the front face 38. The support surface 12 is in electrical communication with a signal processor 18. The support surface 12 is in electrical communication with a computing platform 54 via the signal processor 18. The computing platform 54 includes a user interface 62.

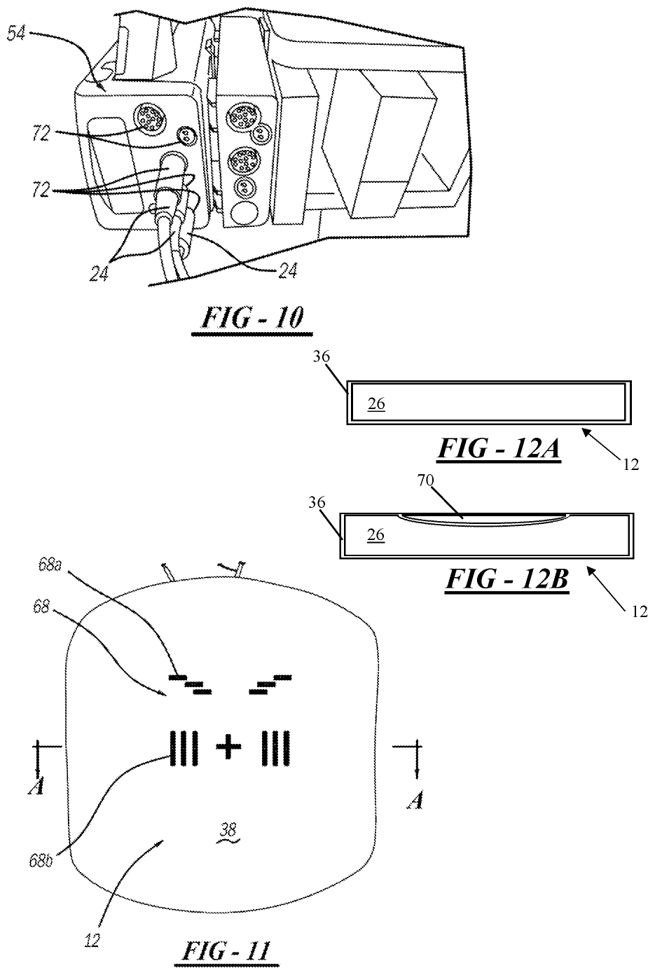

FIG. 10 illustrates a computing platform 54. The computing platform 54 includes a plurality of ports 72. The ports 72 are configured to receive connections from conductors 24.

FIG. 11 illustrates a support surface 12. The support surface 12 may be one compatible with an incubator 66. The support surface 12 includes a front face 38. The support surface 12 includes position indicators 68. The position indicators 68 include shoulder indicators 68a and side indicators 68b. The shoulder indicators 68a may guide placement of the tops of the shoulders of an infant on the front face. The side indicators 68b may guide placement of the sides of an infant's torso on the front face.

FIG. 12A illustrates a cross-section of the support surface 12 taken along section A-A of FIG. 10. The support surface 12 includes an inner layer 26 encapsulated by an outer layer 36.

FIG. 12B illustrates a cross-section of the support surface 12 taken along section A-A of FIG. 10. The support surface 12 includes an inner layer 26 encapsulated by an outer layer 36. The support surface 12 includes a receiving contour 70. The receiving contour 70 is formed in the inner layer 26 and the outer layer 36.

FIG. 13 illustrates a plurality of sensing devices 20 part of a support surface 12. The support surface 12 may find use for use with an infant 104 (not shown). The support surface 12 includes a top face 33 opposing a bottom face 32. The distance between the top face 33 and bottom face 32 is a length L of the support surface 12. The support surface 12 includes opposing side faces 31. The distance between the side faces 31 is a width W of the support surface 12. The support surface 12 includes an inner layer 26. The sensing devices 20 include arm sensing devices 28 and leg sensing devices 30. The arm sensing devices 28 include an upper right arm sensing device 28a, lower right arm sensing device 28b, upper left arm sensing device 28c, and lower left arm sensing device 28d. The leg sensing devices 30 include an upper leg sensing device 30a and a lower leg sensing device 30b.

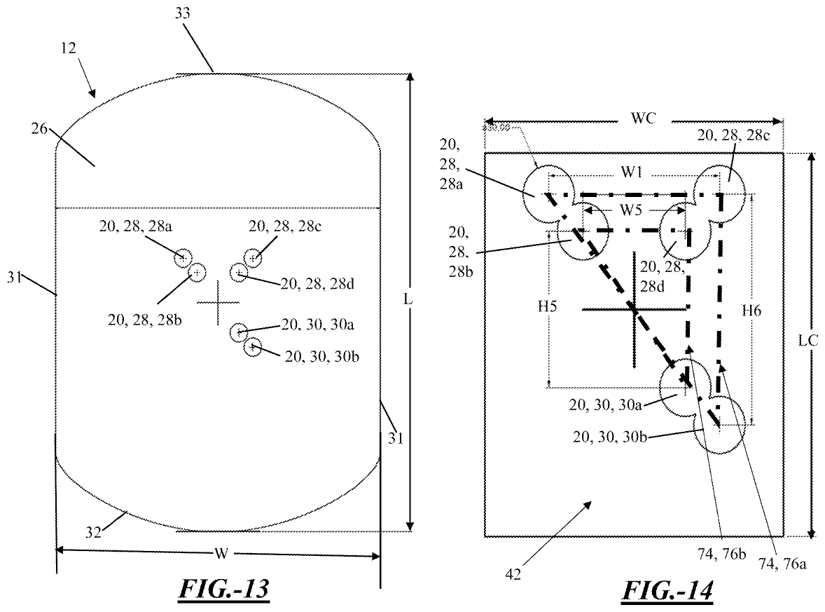

FIG. 14 illustrates a schematic of a conductive sheet 42 with the sensing devices 20 thereon. The conductive sheet 42 has a length LC and width WC. Located on the conductive sheet 42 are the sensing devices 20. The upper right arm sensing device 28a is distanced from the upper left arm sensing device 28c by a width W1. The lower right arm sensing device 28b is distanced from the lower left arm sensing device 28d by a width W5. The lower right arm sensing device 28b and the lower left arm sensing device 28d are distanced from the upper leg sensing device be a height H5. The upper left arm sensing device 28c is distanced from the lower leg sensing device 30b by a height H6. The sensing device 20 are arranged in formations 74. The upper right arm sensing device 28a, upper left arm sensing device 28c, and lower left leg sensing device 30b form an outer shape 76a. The outer shape 76a is in the form of an upside-down triangle. The lower right arm sensing device 28b, lower left arm sensing device 28d, and upper left leg sensing device 30a form an inner shape 76b. The inner shape 76b is in the form of an upside-down triangle. The inner shape 76b is located within the outer shape 76a.

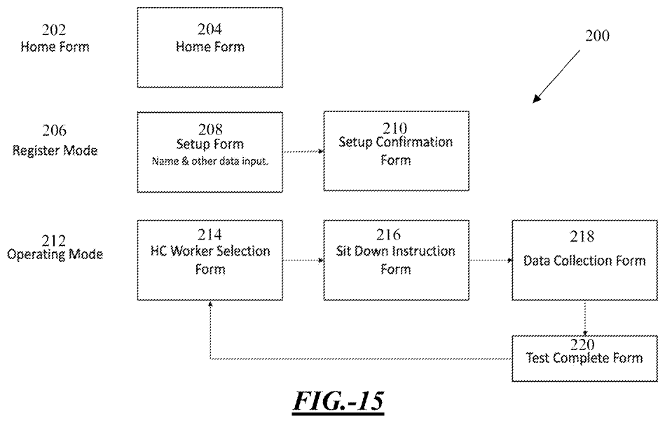

FIG. 15 illustrates a flow chart for usage of a user interface 200. The usage of the user interface is regarding a user interface before or during monitoring a condition of a user (e.g., subject). The user interface may start at a home form 202. The home form may display a home screen on the user interface 204. The home form 202 may direct a user to a registration mode 206, operating mode 212, or both of a system.

The registration mode 206 may function to register one or more users, create one or more user log-ins, save registration data, or a combination thereof to a memory storage device. The registration mode may initiate immediately prior to an operating mode or even multiple days in advance. The registration mode may display a set-up form 208. The set-up form may allow for a user to input one or more user identifiers. The user identifiers may serve to register the user onto the system. The one or more user identifiers may include name (e.g., first, last, middle initial). Once a user inputs their name, the system may compare their data to previously registered to users to ensure the user identifiers are unique. The system may automatically create a username, such as based on first and last initials of a user. After inputting the name, the set-up form may allow for a user to input additional research data. This data may include age, weight, height, race, ethnicity, career, education, known diseases and/or infections, other demographic information, the like, or any combination thereof. Once a user inputs their data, they may confirm completion of data entry. After confirmation of data entry completion, a setup confirmation form may display 210. This may be in the form of a confirmation window on the user interface. The home form, confirmation form, or both may direct a user to an operating mode of the interface.

The operating mode 212 may function to allow one or more individuals to initiate data collection by the system, execution of one or more methods of machine learning and/or detection of one or more diseases, or a combination thereof. The operating mode may initiate with a selection form 214. A selection form 214 may allow for a user to log-in; select a specific user, such as themselves, from previously registered users; or both.

After an individual selects a user via the selection form, the next form may be an instruction form 216. The instruction form may provide instructions or guidance as to how the user should position themselves onto a support surface, relative to one or more sensing devices, or both. For example, the instruction form may display instructions, such as:
“Sit down in chair. Place back flat against the back of the chair. Stay still in the chair until exam is complete.”

The user interface may include a status light separate from a screen, as a backlight of the screen, or an icon in the screen. The status light may show red while an individual is improperly positioned and green once an individual is properly positioned and remaining still on the support surface. A user may also confirm they are properly positioned via a user input (e.g., touchscreen button confirming user is ready). After the system or the user confirms they are properly positioned the instruction form may move on to a data collection form 218.

The data collection form 218 may allow for one or more inputted signals to be displayed during. The one or more inputted signals may include one or more bipotential, vital, and/or other physiological signals associated with the individual. The data collection form may also indicate a timer showing how long may be left until the test is complete. Once sufficient data is collected, a certain amount of time has passed, or both, the user interface may indicate that the test is complete 220. Indication may be by a green light, green screen, words indicating completing, an audible alarm, or any combination thereof. After the test is complete 220, the data collection form closes and a test complete form opens.

The test complete 220 form may communicate to a user that they have completed the monitoring test, are able to step away from a support surface, or both. After the test complete form, the user interface may go back to a home form, a selection form, or both. The test complete form may display the average heart rate, one or more other pieces of collected data, or both.

Any of the forms from registration mode to operation mode may allow for a user to input their health status. For example, the selection form may a field for entry of a current health status of the individual. The field may be a radial button, drop-down list, the like, or any combination thereof.

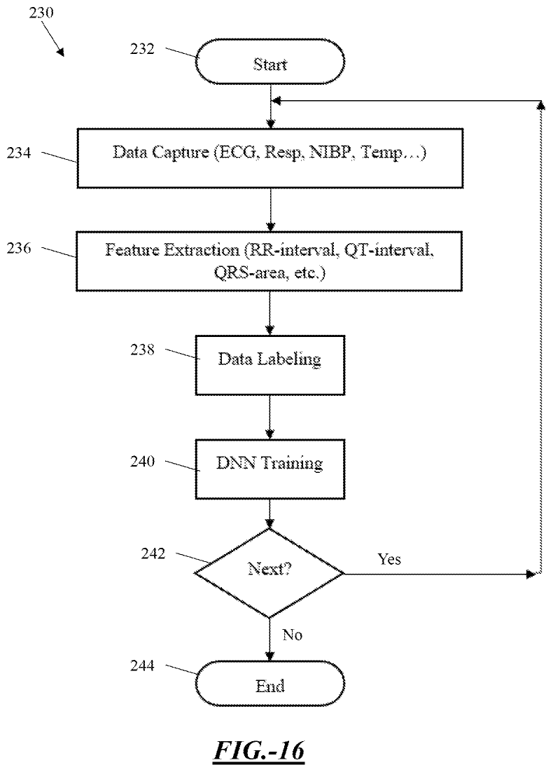

FIG. 16 illustrates a method of machine learning 230. The method may begin 232 by an individual being positioned on a support surface and in proximity to one or more sensing devices part of the system as taught herein. The one or more sensing devices capture one or more signals 234 (e.g., biopotentials, vital signs, and/or other physiological signs). The one or more signals may be saved within one or more databases before or after feature extraction. Feature extraction 236 may be formed on the one or more incoming signals from a user. Feature extraction may include one or more waves, intervals, segments, and/or other waveform morphology characteristics. Feature extraction may include one or more other data points related to vital signs and/or other physiological signs. After feature extraction, this data may be saved in one or more databases. The saved data may be assigned a label during data labeling 238. Data labeling may identify a certain record with a health status of a user. The health status of a user may be the presence and/or absence of a virus or other infection or disease. The data label may be input by a user. For example, the data label may be provided via a user interface during data collection, such as illustrated in FIG. 1. The feature extraction and data labeling may cooperate together during the machine learning stage 240 (e.g., “DNN training”). Machine learning 230 may repeat 242 by capturing data 234 from another individual or come to an end 244 once sufficient data is captured.

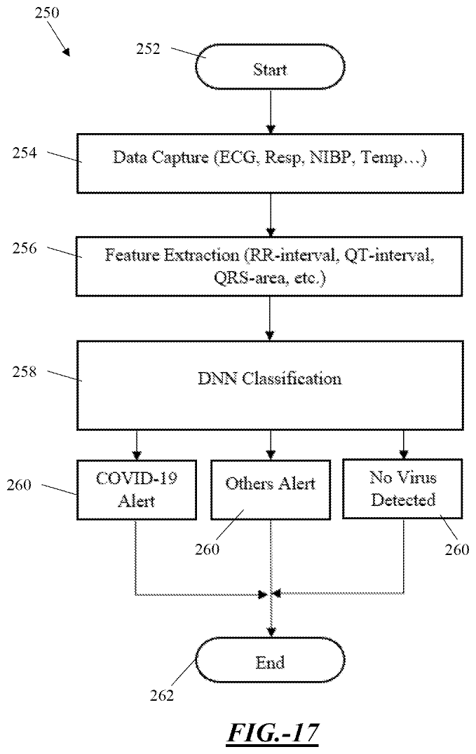

FIG. 17 illustrates a method of identifying a condition 250 of a subject (e.g., human). The method 250 may begin 252 by an individual being positioned on a support surface and in proximity to one or more sensing devices part of the system as taught herein. The one or more sensing devices capture one or more signals 254 (e.g., biopotentials, vital signs, and/or other physiological signs). The one or more signals may be saved within one or more databases before or after feature extraction. One or more features may be extracted 256. These one or more features may have been identified via machine learning. The one or more features may be outputted into a memory storage device, such as a database saved therein. A processor may analyze the one or more features for the presence of one or more biomarkers. Based on the presence of one or more biomarkers, the system is able to identify the presence and/or absence of an infection and/or disease 258, such as COVID-19. After identification, the system is able to alert 260 the user or another professional via one or more user interfaces regarding the health status of an individual.

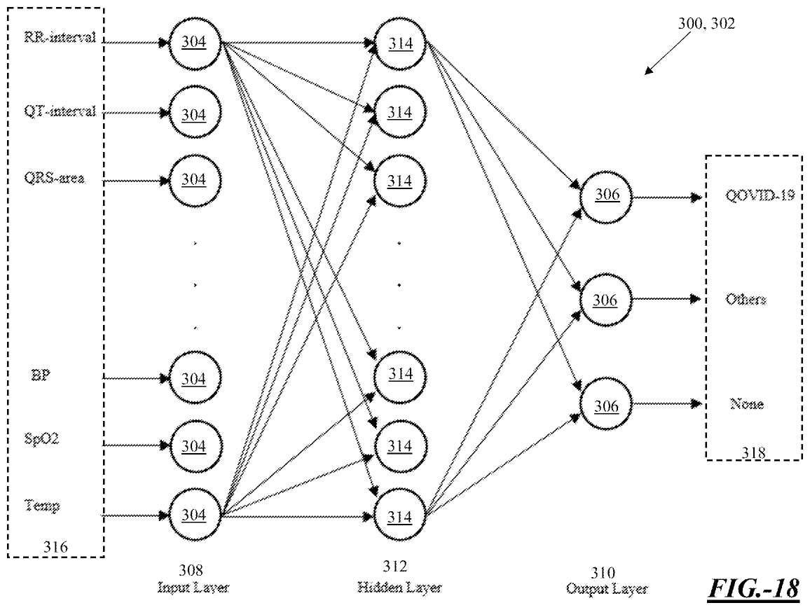

FIG. 18 illustrates a machine learning system network 300. The machine learning system network 300 is illustrated as an artificial neuron network 302. The machine learning system network 300 includes a plurality of input nodes 304 and a plurality of output nodes 306. The input nodes 304 are part of an input layer 308. The output nodes 306 are part of an output layer 310. Between the input layer 308 and output layer 310 is a hidden layer 312. The hidden layer 312 may include a plurality of hidden nodes 314. The machine learning system 300 may receive input for the input nodes 304 as a plurality of parametric values 316 derived from feature extraction. The machine learning system 300 may output a diagnosis 318 based on the output layer 310.

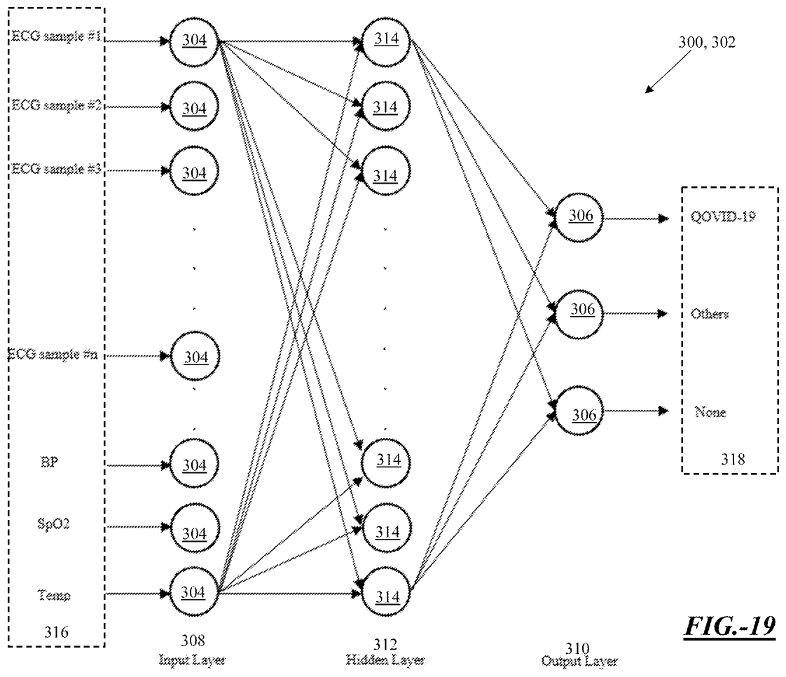

FIG. 19 illustrates a machine learning system network 300. The machine learning system network 300 is illustrated as an artificial neuron network 302. The machine learning system network 300 includes a plurality of input nodes 304 and output nodes 306. The input and output nodes 304, 306 are part of an input layer 308 and output layer 310, respectively. Between the input and output layers 308, 310 is a hidden layer 312. The hidden layer 312 includes a plurality of hidden nodes 314. The machine learning system 300 may receive input as a plurality of both time-series and parametric values 316 derived from time series extraction and feature extraction.

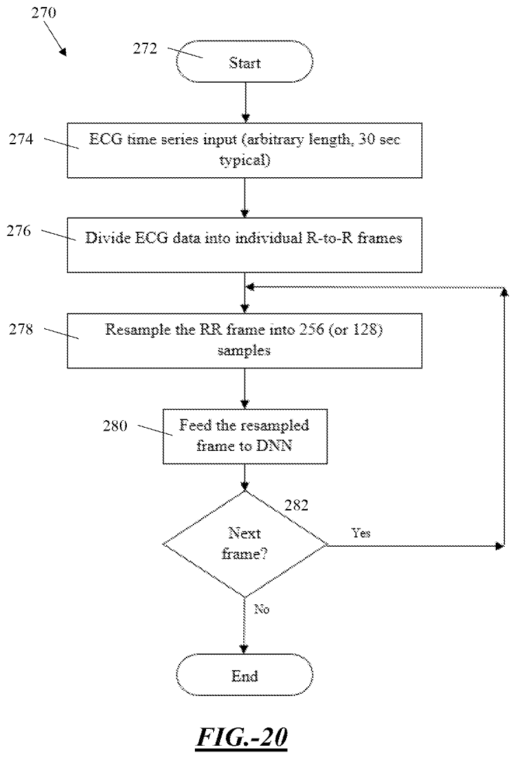

FIG. 20 illustrates a method of preparing data 270 for time-series data extraction and usage by machine learning. The method begins 272 by receiving one or more input signals 274 as a time series input. The one or more input signals may include one or more waveforms associated with one or more biopotentials. The input signals may be taken over a period of time. The period of time may be about 5 seconds or greater, about 15 seconds or greater, or even about 30 seconds or greater. The period of time may be about 5 minutes or less, about 2 minutes or less, or even about 1 minute or less. The collected input signals may be converted into input data. The input data may be divided into individual R-R frames 276. Individual R-R frames may be determined by analyzing the R-R interval of the input waveform. The individual R-R frames may be resampled 278 (e.g., broken down) into as plurality of subsamples. For example, 128 or 256 subsamples. The broken-down samples are fed into the neural network 280. The broken-down samples may be fed into individual input nodes into the neural network. After feeding the broken-down samples of one R-R frame, the method determines 282 if it moves on to a subsequent individual R-R frame or ends 284.

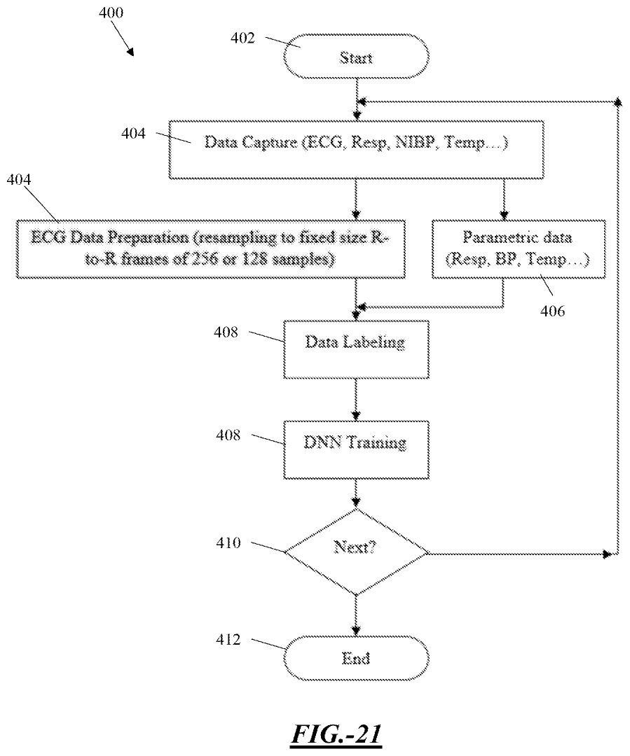

FIG. 21 illustrates a method of machine learning 400. The method 400 may begin by an individual begin 402 positioned on a support surface and in proximity to one or more sensing devices part of the system as taught herein. The one or more sensing devices capture 404 one or more input signals (e.g., biopotentials, vital signs, and/or other physiological signs). The one or more signals may be saved within one or more databases before or after time series data extraction 404, feature extraction 406, or both. Feature extraction may result in parametric data collection 406. Feature extraction may follow a process similar to that as discussed with respect to FIGS. 16-17. Time series data extraction 404 may follow the process as discussed with respect to FIG. 20. After feature extraction 406 and time series extraction 404, the collected data may be assigned a label during data labeling 408. Data labeling 408 may identify a certain record with a health status of a user. The health status of a user may be the presence and/or absence of a virus or other infection or disease. The data label may be input by a user. For example, the data label may be provided via a user interface during data collection, such as illustrated in FIG. 1. The feature extraction and data labeling may cooperate together during the machine learning stage 410 (e.g., “DNN training”). Machine learning 400 may repeat 410 by capturing data 404 from another individual or come to an end 412 once sufficient data is captured.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The terms “generally” or “substantially” to describe angular measurements may mean about +/−10° or less, about +/−5° or less, or even about +/−1° or less. The terms “generally” or “substantially” to describe angular measurements may mean about +/−0.01° or greater, about +/−0.1° or greater, or even about +/−0.5° or greater. The terms “generally” or “substantially” to describe linear measurements, percentages, or ratios may mean about +/−10% or less, about +/−5% or less, or even about +/−1% or less. The terms “generally” or “substantially” to describe linear measurements, percentages, or ratios may mean about +/−0.01% or greater, about +/−0.1% or greater, or even about +/−0.5% or greater.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps. Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

What is claimed is:

1. A contactless system for sensing biopotential signals from a subject comprising:
a support surface including:
a) one or more inner layers including one or more deflecting materials;
b) a plurality of contactless electrode units within the one or more inner layers, the one or more electrode units including one or more capacitive sensors;
c) one or more outer layers located about the one or more inner layers; and
wherein the plurality of contactless electrode units are arranged in an inner shape within an outer shape such that the contactless electrode units form the vertices of the inner shape and the outer shape;
wherein the inner shape is an upside down triangle and wherein the outer shape is an upside down triangle; and
wherein each of the plurality of electrode units includes an amplifier.

2. (canceled)

3. The contactless system of claim 1, wherein the inner shape is formed by three of the plurality of electrode units; and
wherein the outer shape is formed by another three of the plurality of electrode units.

4. The contactless system of claim 3, wherein the inner shape is formed by a lower right arm electrode unit, a lower left arm electrode unit, and an upper left leg electrode unit and
wherein the outer shape is formed by an upper right arm electrode unit, an upper left arm electrode unit, and a lower left leg electrode unit.

5-8. (canceled)

9. The contactless system of claim 1, wherein the one or more outer layers includes one or more permanent layers, temporary layers, or both; and
wherein the plurality of electrode units each have a sensing surface which is flush, above, or below a front surface of the inner layer; and
wherein the sensing surface abuts to one or more outer layers.

10-11. (canceled)

12. The contactless system of claim 1, wherein the one or more inner layers includes one or more conductive sheets;
wherein the one or more conductive sheets is a driven right leg circuit; and
wherein the one or more conductive sheets are located between the one or more deflecting materials and a front face of the one or more outer layers.

13-14. (canceled)

15. The contactless system of claim 1, wherein the support surface is located on or integrated into a secondary support surface; and
wherein the secondary support surface includes a chair, wheelchair, stroller, bed, stretcher, gurney, incubator, floor, table, or a combination thereof.

16. (canceled)

17. The contactless system of claim 15, wherein the chair is the seat of a vehicle and the contactless system is in electrical communication with an electronic control unit (ECU), electronic control module (ECM), onboard diagnostics (OBD), other computing device integrated into the vehicle, or a combination thereof.

18. The contactless system of claim 15, wherein the secondary support surface includes the incubator which is a neonatal incubator; and
wherein the support surface rests on or is integrated into a base surface of the neonatal incubator.

19. (canceled)

20. The contactless system of claim 15, wherein the support surface includes the bed which is a hospital bed, a residential bed, or both.

21. A method of monitoring a condition of a subject which is a human, comprising the steps of:
(a) providing a support surface having one or more sensing devices of an electrocardiograph device embedded therein and/or providing the one or more sensing devices of the electrocardiograph device;
(b) positioning the subject at least partially on the support surface and/or positioning the one or more sensing devices on the subject;
(c) acquiring data from an electrocardiograph reading on the subject for a predetermined amount of time;
(d) outputting the data of the step (c); and
(e) analyzing the data of the step (c), by identifying one or more biomarkers consistent with a medical condition, wherein the analyzing the data is performed by at least one processor; and,
wherein the analyzing step (e) includes a step of comparing at least a portion of the data acquired in the step (c) with pre-existing biomarkers data representative of the medical condition; and
wherein the pre-existing biomarker data representative of the medical condition includes pre-existing data for at least one biomarker selected from QT segment elongation, T-wave depression, and R to R interval.

22. (canceled)

23. (canceled)

24. The method of claim 21, wherein the pre-existing biomarker data representative of the medical condition includes pre-existing data for at least three biomarkers.

25. The method of claim 21, wherein the pre-existing biomarker data is representative of a virus.

26. The method of claim 21, wherein the method is performed at least 12 hours before the onset of a fever and/or other physical symptoms of the subject for detecting the presence of the medical condition.

27. The method of claim 21, wherein the method includes repeating the steps (a)-(c) one or more times and comparing at least a portion of the data of successive performances of those steps.

28. The method of claim 21, wherein the method includes storing at least a portion of the data acquired from the electrocardiograph reading from one or more sets of subjects, and such stored data is used for performing the step (e) for one or more subsequent sets of subjects.

29. (canceled)

30. The method of claim 21, wherein the analyzing step (e) includes a step of analyzing results of at least one other medical sensing device; and
wherein the at least one other medical sensing device includes a sphygmomanometer, a spirometer, an acoustic blood pressuring device, an imaging unit, a capnography monitor, a pulse oximeter, or any combination thereof.

31. (canceled)

32. The method of claim 21, wherein the method is performed by a contactless system for sensing biopotential signals from the subject, the contactless system comprising;
a support surface including;
a) one or more inner layers including one or more deflecting materials;
b) a plurality of contactless electrode units within the one or more inner layers, the one or more electrode units including one or more capacitive sensors;
c) one or more outer layers located about the one or more inner layers; and
wherein the plurality of contactless electrode units are arrange in an inner shape within an outer shape such that the contactless electrode units form the vertices of the inner shape and the outer shape;
wherein the inner shape is an upside down triangle and wherein the outer shape is an upside down triangle.

33-43. (canceled)

44. A method of machine learning to identify one or more biomarkers indicative of a medical condition comprising:
(a) acquiring data from an electrocardiograph reading on a subject which is human for a predetermined amount of time;
(b) outputting the data of the step (a);
(c) applying one or more data labels identifying a health status of the subject; and
(d) analyzing the data to find a correlation between the data and the one or more data labels to determine the one or more biomarkers indicative of the medical condition.

45-46. (canceled)

47. The method of claim 44, wherein the step of outputting the data includes a time series extraction, a feature extraction, or both.

48. The method of claim 45, wherein the method includes providing a support surface having one or more sensing devices of an electrocardiograph embedded therein;
wherein the method includes positioning the subject at least partially on the support surface; and
wherein the support surface comprises;
a) one or more inner layers including one or more deflecting materials;
b) a plurality of contactless electrode units within the one or more inner layers, the one or more electrode units including one or more capacitive sensors;
c) one or more outer layers located about the one or more inner layers; and
wherein the plurality of contactless electrode units are arrange in an inner shape within an outer shape such that the contactless electrode units form the vertices of the inner shape and the outer shape; wherein the inner shape is an upside down triangle and wherein the outer shape is an upside down triangle.

49-107. (canceled)
